(19) United States
(12) Patent Application Publication
(10) Pub. No.: US 2021/0225457 A1
(43) Pub. Date: Jul. 22, 2021

(54) METHODS FOR GENERATING PAN-EPITOPIC IMMUNOGENS OF INFLUENZA H3 VIRUS, COMPOSITIONS AND METHODS OF USE THEREOF

(71) Applicant: University of Georgia Research Foundation, Athens, GA (US)

(72) Inventors: James Daniel Allen, Athens, GA (US); Ted Milburn Ross, Athens, GA (US); Terianne Maiko Wong, Athens, GA (US)

(73) Assignee: University of Georgia Research Foundation, Athens, GA (US)

(21) Appl. No.: 17/259,885

(22) PCT Filed: Jul. 12, 2019
(86) PCT No.: PCT/US2019/041710

§ 371 Date: Jan. 12, 2021

Related U.S. Application Data
(60) Provisional application No. 62/697,803, filed on Jul. 13, 2018.

(51) Int. Cl. G16B 40/00; A61K 39/145; A61P 31/16; C12N 7/00

(52) U.S. Cl. CPC G16B 40/00; A61K 39/145; A61P 31/16; C12N 7/00; C12N 2760/16122; C12N 2760/16134; C12N 2760/16171; C12N 2760/16121; C12N 2760/16123

(57) ABSTRACT

Provided herein are methods for generating a non-naturally occurring, broadly reactive, pan-epitopic antigen derived from H3 influenza virus that is capable of eliciting a broadly reactive immune response, such as a broadly reactive neutralizing antibody response, against H3 virus following administration to a subject. Also provided is a non-naturally occurring immunogen generated using the methods, and vaccines and compositions comprising the immunogen. Methods of generating an immune response in a subject by administering the immunogen, vaccine, or composition are provided. In particular, the immunogen comprises the hemagglutinin (HA) protein of H3 influenza vims strains.

### CROSS-REFERENCE TO RELATED APPLICATION

This application claims priority to and benefit of U.S. Provisional Application No. 62/697,803, filed on Jul. 13, 2018, the entire contents of which are incorporated by reference herein in their entirety.

### BACKGROUND

In 2017, the U.S. Centers for Disease Control and Prevention estimated that the seasonal flu vaccine was only 42% effective. This limited effectiveness was due to a mutation that occurred in the influenza A (H3N2) vaccine strain that causes flu in infected individuals. In addition, cases of flu caused by influenza B viruses have risen in the 2017 to 2018 time period. Given that a bad flu season can kill on the order of 50,000 people in the United States alone, new and improved methods for generating vaccines that could provide broad protection against viruses, particularly, influenza A H3N2 and influenza B virus strains, in present and future circulation are urgently required.

### SUMMARY OF THE DISCLOSURE

As described below, methods of generating synthetic (non-naturally occurring), broadly reactive antigens and antigen sequences derived from the Influenza A H3 virus (also referred to as “H3 influenza,” “H3 influenza virus,” “H3 virus,” or simply “H3” herein), such as subtype H3N2, are provided. These H3 virus antigens include structural proteins or peptides, in particular, the hemagglutinin (HA) protein, or the HA1 (head) or HA2 (tail or stalk) portions of the HA protein, and are potent immunogens that can elicit a broadly reactive immune response against H3 HA protein and, ultimately, against present and future H3 virus strains in a subject. As referred to herein, the H3 virus antigens or antigen sequences that are generated by the methods and that elicit an immune response in a subject are immunogenic antigens or immunogens. These H3 immunogens are termed broadly reactive and pan-epitopic, because they elicit the production of broadly reactive antibodies that are directed against different subtypes or strains of H3 viruses having both sequence similarity and variability, and a diversity of epitopes (antigenic determinants) in their antigens and sequences thereof, particularly, the HA antigen.

Also provided are immunogenic compositions that contain the broadly reactive H3 antigen, including vaccines (e.g., polypeptide or polynucleotide products), that induce an immune response directed against H3 in a subject; and methods of using the immunogens to induce an immune response in a subject. In a particular embodiment, the antigen is the HA, HA1 or HA2 protein of H3 influenza virus. In a certain embodiment, the antigen is the HA, HA1 or HA2 protein of the H3N2 subtype of influenza virus. Methods of using the immunogens to induce an immune response in a subject are also provided.

In an aspect, a method of generating a non-naturally occurring, pan-epitopic immunogen for generating an immune response against present and future H3 influenza virus strains in a subject is provided, in which the method comprises (a) generating a phylogenetic tree comprising full length related antigen sequences derived from H3 strains present during two or more consecutive flu seasons in the Northern and Southern Hemispheres; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; (f) generating a phylogenetic tree comprising the secondary sequences, wherein branches of the tree are combined if the substitution rate per amino acid site distance is less than about 0.001 to produce a plurality of tertiary sequences; (g) generating a quaternary backbone sequence comprising amino acids that are conserved or identical among the tertiary sequences; and (h) generating a non-naturally occurring, pan-epitopic immunogen by incorporating secondary sequences from step (e), wherein the selected sequences are derived from the most recent time period. In an embodiment of the method, in step (h), the most recent time period is a 6-month time period. In an embodiment of the method, step (d) and step (e) are optional. In an embodiment of the method, steps (a)-(c) are repeated two or more times. In an embodiment of the method, steps (b) and (c) are repeated two or more times prior to step (d). In an embodiment of the method, steps (e) and (f) are repeated two or more times.

It will be understood that the methods described herein generate a non-naturally occurring, pan-epitopic immunogen, which may be referred to interchangeably herein, as a “non-naturally occurring immunogen,” a “broadly reactive immunogen,” or a “pan-epitopic immunogen,” for simplicity.

In an embodiment of the method of the above-delineated aspects, the method further comprises generating at least 2 H3 secondary antigen sequences for a disease season of time based on the phylogenetic tree sequences generated in step (f); adding the at least 2 H3 secondary sequences to a backbone sequence based on clustered antigen sequences in the season; eliminating from the backbone sequence older secondary antigen sequences produced from a predetermined disease season in a geographic location; and generating a pan-epitopic H3 immunogen by equally weighting multiple secondary antigen sequences. In an embodiment, the antigen sequences are selected from H3 influenza virus HA, HA1 or HA2 antigen sequences.

In an embodiment of the method of the above-delineated aspects, the method further comprises generating at least 2 H3 secondary antigen sequences for a disease season of time based on the phylogenetic tree sequences generated in step (f); adding the at least 2 H3 secondary sequences to a backbone sequence based on clustered antigen sequences in the season; retaining in the backbone sequence antigen sequences produced from a predetermined disease season in a geographic location to include an additional season; and generating a pan-epitopic H3 immunogen by equally weighting multiple secondary antigen sequences. In an embodiment, the antigen sequences are selected from H3 influenza virus HA, HA1 or HA2 antigen sequences.

In another aspect, method of generating a non-naturally occurring, pan-epitopic immunogen for generating an immune response against present and future H3 Influenza virus strains in a subject is provided, in which the method comprises (a) generating a phylogenetic tree comprising full length related antigen sequences derived from H3 strains present during two or more consecutive recent flu seasons in the Northern and Southern Hemispheres within a six-month time period; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; (f) repeating steps (a)-(e) until secondary sequences from a series of recent consecutive six-month time periods have been selected over a preselected total time period; (g) generating a phylogenetic tree comprising the secondary sequences, wherein branches of the tree are combined if the substitution rate per amino acid site distance is less than about 0.001 to produce a plurality of tertiary sequences; (h) generating a quaternary backbone sequence comprising amino acids that are conserved or identical among the tertiary sequences; and (i) generating a non-naturally occurring, pan-epitopic immunogen by incorporating secondary sequences from step (e) into the quaternary backbone sequence, wherein (i) secondary sequences from the most recent six-month time period are incorporated into the backbone sequence and secondary sequences from the oldest six-month time period are eliminated from the backbone sequence, thereby producing a sequence comprising multiple secondary sequences spanning the preselected total time period; or (ii) secondary sequences from the most recent six-month time period and from the oldest six-month time period are incorporated into the backbone sequence, thereby producing a sequence comprising multiple secondary sequences spanning the preselected total time period. In an embodiment of the method, the preselected total time period is 2.5 years.

In an embodiment of the methods of any of the above-delineated aspects or any aspect herein, the phylogenetic tree comprises sequences derived from influenza virus strains present during the past 1-100 years. In another embodiment, the phylogenetic tree comprises sequences derived from influenza virus strains present during the past 5, 10, 20, 30, 40, 50, or 100 years.

In an embodiment of the methods of any of the above-delineated aspects or any aspect herein, three or more clusters of antigen sequences are identified in step (b). In a particular embodiment, 5 or 15 clusters of antigen sequences are identified in step (b).

In an embodiment of the methods of any of the above-delineated aspects or any aspect herein, the antigen sequences are H3 influenza virus HA, HA1 or HA2 antigen sequences. In an embodiment of the method, the virus is present in the Northern Hemisphere. In another embodiment of the method, the virus is present in the Southern Hemisphere.

In an embodiment of the methods of any of the above-delineated aspects or other aspects herein, the immunogen generated by the practice of the methods is isolated and/or purified. In another embodiment of the methods, the immunogen is formulated for administration to a subject in need thereof. In an embodiment of the methods, the immunogen or a composition thereof is administered to a subject in need thereof in an effective amount to elicit an immune response in the subject. In an embodiment of the methods, the immune response elicits neutralizing antibodies. In an embodiment of the methods, the immune response elicits a T-lymphocyte response. In an embodiment, the immune response is prophylactic or therapeutic.

In an embodiment, the methods of any of the above-delineated aspect or other aspects herein further comprise the step of displaying or translating the results or output of the steps in a visual form. In an embodiment, the results or output comprise primary, secondary and tertiary sequences, sequence clusters, sequence alignments, phylogenetic trees, or any combinations thereof. In an embodiment, the step of displaying or translating includes displaying the results or output on a display device. In an embodiment, display device is a desktop computer, a laptop computer, a hand-held computer, a smart phone, a cellular telephone, a tablet computer, or a personal digital assistant.

In another aspect, a non-naturally occurring immunogen is provided, wherein the non-naturally occurring immunogen is generated using the methods of the above-delineated aspects and as described supra and infra.

In another aspect, an immunogenic composition or vaccine comprising the non-naturally occurring immunogen generated using the methods of the above-delineated aspects and as described supra and infra is provided.

In another aspect, an influenza virus-like particle (VLP) comprising the non-naturally occurring immunogen, or sequence thereof, generated using the methods of the above-delineated aspects and as described supra and infra is provided. In an embodiment, a vaccine comprising the VLP is provided.

In another aspect, a composition comprising the immunogen, vaccine, or VLP of any of the foregoing aspects and a pharmaceutically acceptable carrier is provided. In an embodiment, the composition further comprises an adjuvant.

In another aspect, a method of generating or eliciting an immune response in a subject is provided, in which the method comprises administering to the subject an effective amount of a non-naturally occurring immunogen generated using the methods of the above-delineated aspects and as described supra and infra. In an embodiment, the immunogen is provided as a vaccine, as a VLP, or in a composition comprising a pharmaceutically acceptable carrier, diluent, or excipient. In an embodiment, an adjuvant is concomitantly administered to the subject.

In an embodiment of the methods of the above aspects, the full length related antigen sequence derived from the first H3 strains selected is deleted from the analysis each time an H3 strain selected from a later flu season is added to the analysis.

In an embodiment of the method of the above aspects, steps a-g are repeated a plurality of times, such that in each repetition full length related antigen sequences are derived from a set of H3 strains present during a flu season that is one season later in time than the flu seasons of the first set of H3 strains selected for analysis.

In an embodiment of the method of the above aspects, steps a-g are repeated a plurality of times, such that in each repetition a full length related antigen sequence is derived from a set of H3 strains present during consecutive flu seasons up to the most recent flu season selected for analysis.

### Definitions

Unless defined otherwise, all technical and scientific terms used herein have the meaning commonly understood by a person skilled in the art to which this invention pertains or relates. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); Benjamin Lewin, Genes V, published by Oxford University Press, 1994 (ISBN 0-19-854287-9); Kendrew et al. (eds.); The Encyclopedia of Molecular Biology, published by Blackwell Science Ltd., 1994 (ISBN 0-632-02182-9); Molecular Biology and Biotechnology: a Comprehensive Desk Reference, Robert A. Meyers (ed.), published by VCH Publishers, Inc., 1995 (ISBN 1-56081-569-8); and Hale & Marham, The Harper Collins Dictionary of Biology (1991). As used herein, the following terms have the meanings ascribed to them below, unless specified otherwise.

By “adjuvant” is meant a substance or vehicle that non-specifically enhances the immune response to an antigen. Adjuvants may include a suspension of minerals (e.g., alum, aluminum hydroxide, or phosphate) on which antigen is adsorbed; or water-in-oil emulsion in which antigen solution is emulsified in mineral oil (e.g., Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (see, e.g., U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. Exemplary biological adjuvants include, without limitation, interleukin-1 (IL-2), the protein memory T-cell attractant “Regulated on Activation, Normal T Expressed and Secreted” (RANTES), granulocyte-macrophage-colony stimulating factor (GM-CSF), tumor necrosis factor-alpha (TNF-α), interferon-gamma (IFN-γ), granulocyte-colony stimulation factor (G-CSF), lymphocyte function-associated antigen 3 (LFA-3, also called CD58), cluster of differentiation antigen 72 (CD72), (a negative regulator of B cell responsiveness), peripheral membrane protein, B7-1 (B7-1, also called CD80), peripheral membrane protein, B7-2 (B7-2, also called CD86), the TNF ligand superfamily member 4 ligand (OX40L) or the type 2 transmembrane glycoprotein receptor belonging to the TNF superfamily (4-1BBL)

By “administer” is meant giving, supplying, dispensing a composition, agent, therapeutic and the like to a subject, or applying or bringing the composition and the like into contact with the subject. Administering or administration may be accomplished by any of a number of routes, such as, for example, without limitation, topical, oral, subcutaneous, intramuscular, intraperitoneal, intravenous (IV), (injection), intrathecal, intramuscular, dermal, intradermal, intracranial, inhalation, rectal, intravaginal, or intraocular.

By “agent” is meant any small molecule chemical compound, antibody, nucleic acid molecule, peptide, polypeptide, or fragments thereof.

By “alteration” is meant a change (increase or decrease) in the expression levels or activity of a gene or polypeptide as detected by standard art known methods such as those described herein. As used herein, an alteration includes a 5% change in expression levels, a 10% change in expression levels, preferably a 25% change, more preferably a 40% change, and most preferably a 50% or greater change in expression levels.”

By “ameliorate” is meant decrease, reduce, diminish, suppress, attenuate, arrest, or stabilize the development or progression of a disease or pathological condition.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “antibody” is meant an immunoglobulin (Ig) molecule produced by B lymphoid cells and having a specific amino acid sequence. Antibodies are evoked or elicited in subjects (humans or other animals or mammals) following exposure to a specific antigen (immunogen). A subject capable of generating antibodies/immunoglobulin (i.e., an immune response) directed against a specific antigen/immunogen is said to be immunocompetent. Antibodies are characterized by reacting specifically with (e.g., binding to) an antigen or immunogen in some demonstrable way, antibody and antigen/immunogen each being defined in terms of the other.

“Eliciting an antibody response” refers to the ability of an antigen, immunogen or other molecule to induce the production of antibodies. Antibodies are of different classes, e.g., IgM, IgG, IgA, IgE, IgD and subtypes or subclasses, e.g., IgG1, IgG2, IgG2a, IgG2b, IgG3, IgG4. An antibody/immunoglobulin response elicited in a subject can neutralize a pathogenic (e.g., infectious or disease-causing) agent by binding to epitopes (antigenic determinants) on the agent and blocking or inhibiting the activity of the agent, and/or by forming a binding complex with the agent that is cleared from the system of the subject, e.g., via the liver.

As used herein, “broadly reactive” means that an immune response is elicited against a viral protein (e.g., a virus protein sequence) in a subject that is sufficient to block, inhibit, impede, neutralize, or prevent infection of a broad range of related influenza viruses (such as most or all influenza viruses within a specific subtype).

By “antigen” is meant a compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. In some embodiments of the disclosed compositions and methods, the antigen is an influenza hemagglutinin (HA) protein. In many cases, an antigen that elicits or stimulates an immune response in a subject is termed an “immunogen.”

The term “antigenic drift” refers to a mechanism for variation in organisms or microorganisms such as viruses that involves the accumulation of mutations within the genes that code for antibody-binding sites (also called antigenic determinants or epitopes). This process results in a new strain of virus/virus particles that is not inhibited or blocked as effectively by antibodies that were originally generated against the antigens of virus strains prior to mutation, thus allowing the virus to spread more easily throughout a partially immune population. By way of example, antigenic drift occurs in both influenza A and influenza B viruses.

In the context of a live virus, the term “attenuated” reflects a virus that is attenuated if its ability to infect a cell or subject and/or its ability to produce disease is reduced (for example, diminished, abrogated, or eliminated) compared to the ability of a wild-type virus to produce disease in the subject. Typically, an attenuated virus retains at least some capacity to elicit an immune response following administration to an immunocompetent subject. In some cases, an attenuated virus can elicit a protective immune response without causing any signs or symptoms of infection. In some embodiments, the ability of an attenuated virus to cause disease or pathology in a subject is reduced at least about or equal to 5%, or at least about or equal to 10%, or at least about or equal to 25%, at least about or equal to 50%, at least about or equal to 75%, or at least about or equal to 80%, or at least about or equal to 85%, or at least about or equal to 90%, or at least about or equal to 95%, or greater, relative to the ability of a wild-type virus to cause disease or pathology in the subject.

The term “clade” refers to the different categorizations (often called subtypes) of the known influenza viruses, such as, e.g., the influenza A H3N2 virus. Viruses in an H3N2 clade are genetically related, but do not share the exact viral genome. By way of example, there are many different clades of H3N2 virus subtypes designated in the art. One clade is 3C.2a, and subclades include 3C.2a.1, 3C.2a.2, 3C.2a.3 and 3C.2a.4. In addition, there are at least ten different clades of H5N1 virus subtypes designated in the art: clade 0 clade 1, clade 2, clade 3, clade 4, clade 5, clade 6, clade 7, clade 8 and clade 9 (Abdel-Ghafar et al., N Engl J Med 358:261-273, 2008). Clade 2 is further divided into sub-clades (including clade 2.1, clade 2.2, clade 2.3, clade 2.4 and clade 2.5).

A “codon-optimized” nucleic acid refers to a nucleic acid sequence that has been altered such that the codons are optimal for expression in a particular system (such as a particular species of group of species). For example, a nucleic acid sequence can be optimized for expression in mammalian cells. Codon optimization does not alter the amino acid sequence of the encoded protein.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of an analyte, compound, agent, or substance to be detected. By “detectable label” is meant a composition that, when linked to a molecule of interest, renders the latter detectable, e.g., via spectroscopic, photochemical, biochemical, immunochemical, or chemical means. Nonlimiting examples of useful detectable labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition, disorder, or pathology that damages or interferes with the normal function of a cell, tissue, or organ. Examples of diseases include those caused by H3 virus infection and the symptoms and adverse effects that are caused by infection of the body with the H3 virus. Influenza virus causes flu and its symptoms in infected individuals.

By “effective amount” is meant the amount of an active therapeutic agent, composition, compound, biologic (e.g., a vaccine or therapeutic peptide, polypeptide, or polynucleotide) required to ameliorate, reduce, improve, abrogate, diminish, or eliminate the symptoms and/or effects of a disease, condition, or pathology relative to an untreated patient. The effective amount of an immunogen or a composition comprising an immunogen, as used to practice the methods of therapeutic treatment of disease, condition, or pathology caused by the H3 virus, varies depending upon the manner of administration, the age, body weight, and general health of the subject. Ultimately, the attending physician or veterinarian will decide the appropriate amount and dosage regimen. Such amount is referred to as an “effective” amount.

A “therapeutically effective amount” refers to a quantity of a specified agent sufficient to achieve a desired effect in a subject being treated with that agent. For example, this may be the amount of an H3 influenza virus immunogen or vaccine useful for eliciting an immune response in a subject and/or for preventing infection by H3 influenza virus. Ideally, in the context of the present disclosure, a therapeutically effective amount of an influenza vaccine or immunogenic composition is an amount sufficient to increase resistance to, prevent, ameliorate, reduce, and/or treat infection caused by influenza virus in a subject without causing a substantial cytotoxic effect in the subject. The effective amount of an influenza vaccine useful for increasing resistance to, preventing, ameliorating, reducing, and/or treating infection in a subject depends on, for example, the subject being treated, the manner of administration of the therapeutic composition and other factors, as noted supra.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. A portion or fragment of a polypeptide may be a peptide. In the case of an antibody or immunoglobulin fragment, the fragment typically binds to the target antigen.

By “fusion protein” is meant a protein generated by expression of a nucleic acid (polynucleotide) sequence engineered from nucleic acid sequences encoding at least a portion of two different (heterologous) proteins or peptides. To create a fusion protein, the nucleic acid sequences must be in the same reading frame and contain no internal stop codons. For example, a fusion protein includes an H3 influenza HA protein fused to a heterologous protein.

By “genetic vaccine” is meant an immunogenic composition comprising a polynucleotide encoding an antigen.”

The terms “geographical location or geographical region” refers to preselected divisions of geographical areas of the earth, for example, by continent or other preselected territory or subdivision (e.g., the Middle East, which spans more than one continent). Examples of different geographical regions include countries (e.g., Turkey, Egypt, Iraq, Azerbaijan, China, United States); continents (e.g., Asia, Europe, North America, South America, Oceania, Africa); recognized geopolitical subdivisions (such as the Middle East); or hemispheres of the world (e.g., Northern, Southern, Eastern, or Western hemispheres).

The term “Hemagglutinin (HA)” refers to a surface glycoprotein expressed by an influenza virus. HA mediates binding of the virus particle to a host cell and subsequent entry of the virus into the host cell. The nucleotide and amino acid sequences of numerous influenza HA proteins are known in the art and are publically available, such as those deposited with GenBank, see, e.g., U.S. Publication No. US 2015/0030628, Table 1). HA (along with neuraminidase (NA)) is one of the two major influenza virus antigenic proteins having antigenic determinants (epitopes) that are recognized and bound by antibodies/immunoglobulins.

By way of example, a hemagglutinin (HA) protein of an influenza H3N2 virus is a polypeptide or fragment thereof having at least about or equal to 85%, or at least about or equal to 90%, 95%, 98%, 99%, or greater, amino acid sequence identity to the amino acid sequence of Influenza A virus (A/Hong Kong/1-4/1968(H3N2)) segment 4, complete sequence, Accession Number CY033017, as set forth below:

MKTIIALSYIFCLALGQDLPGNDNSTATLCLGHHAVPNGTLVKTITDDQI
EVTNATELVQSSSTGKICNNPHRILDGIDCTLIDALLGDPHCDVFQNETW
DLFVERSKAFSNCYPYDVPDYASLRSLVASSGTLEFITEGFTWTGVTQNG
GSNACKRGPGSGFFSRLNWLTKSGSTYPVLNVTMPNNDNFDKLYIWGVHH
PSTNQEQTSLYVQASGRVTVSTRRSQQTIIPNIGSRPWVRGLSSRISIYW
TIVKPGDVLVINSNGNLIAPRGYFKMRTGKSSIMRSDAPITCISECITPN
GSIPNDKPFQNVNKITYGACPKYVKQNTLKLATGMRNVPEKQTRGLFGAI
AGFIENGWEGMIDGWYGFRHQNSEGTGQAADLKSTQAAIDQINGKLNRVI
EKTNEKFHQIEKEFSEVEGRIQDLEKYVEDTKIDLWSYNAELLVALENQH
TIDLTDSEMNKLFEKTRRQLRENAEDMGNGCFKIYHKCDNACIESIRNGT
YDHDVYRDEALNNRFQIKGVELKSGYKDWILWISFAISCFLLCVVLLGFI
MWACQRGNIRCNICI.

In addition, a hemagglutinin (HA) protein of an influenza H3N2 virus is encoded by a polynucleotide or fragment thereof having at least about or equal to 85%, or at least about or equal to 90%, 95%, 98%, 99%, or greater, sequence identity to the polynucleotide sequence as follows:

| | |
|---|---|
| 1 | attaatcatg aagaccatca ttgctttgag ctacattttc tgtctggctc tcggccaaga |
| 61 | ccttccagga aatgacaaca gcacagcaac gctgtgcctg ggacatcatg cggtgccaaa |
| 121 | cggaacacta gtgaaaacaa tcacagatga tcagattgaa gtgactaatg ctactgagct |
| 181 | agttcagagc tcctcaacgg ggaaaatatg caacaatcct catcgaatcc ttgatggaat |
| 241 | agactgcaca ctgatagatg ctctattggg ggaccctcat tgtgatgttt ttcaaaatga |
| 301 | gacatgggac cttttcgttg aacgcagcaa agctttcagc aactgttacc cttatgatgt |
| 361 | gccagattat gcctccctta ggtcactagt tgcctcgtca ggcactctgg agtttatcac |
| 421 | tgagggtttc acttggactg gggtcactca gaatggggga agcaatgctt gcaaaagggg |
| 481 | acctggtagc ggttttttca gtagactgaa ctggttgacc aaatcaggaa gcacatatcc |
| 541 | agtgctgaac gtgactatgc caaacaatga caattttgac aaactataca tttggggggt |
| 601 | tcaccacccg agcacgaacc aagaacaaac cagcctgtat gttcaagcat cagggagagt |
| 661 | cacagtctct accaggagaa gccagcaaac tataatcccg aatatcgggt ccagaccctg |
| 721 | ggtaaggggt ctgtctagta gaataagcat ctattggaca atagttaagc cgggagacgt |
| 781 | actggtaatt aatagtaatg ggaacctaat cgctcctcgg ggttatttca aaatgcgcac |
| 841 | tgggaaaagc tcaataatga ggtcagatgc acctattgat acctgtattt ctgaatgcat |
| 901 | cactccaaat ggaagcattc ccaatgacaa gccctttcaa aacgtaaaca agatcacata |
| 961 | tggagcatgc cccaagtatg ttaagcaaaa caccctgaag ttggcaacag ggatgcggaa |
| 1021 | tgtaccagag aaacaaacta gaggcctatt cggcgcaata gcaggtttca tagaaaatgg |
| 1081 | ttgggaggga atgatagacg gttggtacgg tttcaggcat caaaattctg agggcacagg |
| 1141 | acaagcagca gatcttaaaa gcactcaagc agccatcgac caaatcaatg ggaaattgaa |
| 1201 | cagggtaatc gagaagacga acgagaaatt ccatcaaatc gaaaaggaat tctcagaagt |
| 1261 | agaagggaga attcaggacc tcgagaaata cgttgaagac actaaaatag atctctggtc |
| 1321 | ttacaatgcg gagcttcttg tcgctctgga gaatcaacat acaattgacc tgactgactc |
| 1381 | ggaaatgaac aagctgtttg aaaaaacaag gaggcaactg agggaaaatg ctgaagacat |
| 1441 | gggcaatggt tgcttcaaaa tataccacaa atgtgacaac gcttgcatag agtcaatcag |
| 1501 | aaatgggact tatgaccatg atgtatacag agacgaagca ttaaacaacc ggtttcagat |
| 1561 | caaaggtgtt gaactgaagt ctggatacaa agactggatc ctgtggattt cctttgccat |
| 1621 | atcatgcttt ttgctttgtg ttgttttgct ggggttcatc atgtgggcct gccagagagg |
| 1681 | caacattagg tgcaacattt gcatttgagt gtattagtaa |

“Hybridization” means hydrogen bonding, which may be Watson-Crick, Hoogsteen, or reversed Hoogsteen hydrogen bonding, between complementary nucleobases. For example, in DNA, adenine and thymine, and cytosine and guanine, are, respectively, complementary nucleobases that pair through the formation of hydrogen bonds.

The term “immune response” is meant any response mediated by an immunoresponsive cell. In one example of an immune response, leukocytes are recruited to carry out a variety of different specific functions in response to exposure to an antigen (e.g., a foreign entity). Immune responses are multifactorial processes that differ depending on the type of cells involved. Immune responses include cell-mediated responses (e.g., T cell responses), humoral responses (B cell/antibody responses), innate responses and combinations thereof.

By “immunogen” is meant a compound, composition, or substance which, under appropriate conditions, can elicit or stimulate an immune response, such as the production of antibodies, and/or a T-cell response, in an animal, including compositions that are injected or absorbed into an animal. As used herein, an “immunogenic composition” is a composition comprising an immunogen (such as an H3 HA polypeptide or polynucleotide encoding said polypeptide) or a vaccine comprising an H3 HA polypeptide or polynucleotide encoding said polypeptide). As will be appreciated by the skilled person in the art, if administered to a subject in need prior to the subject's contracting disease or experiencing full-blown disease, an immunogenic composition can be prophylactic and result in the subject's eliciting an immune response, e.g., a neutralizing antibody and/or cellular immune response, to protect against disease, or to prevent more severe disease or condition, and/or the symptoms thereof. If administered to a subject in need following the subject's contracting disease, an immunogenic composition can be therapeutic and result in the subject's eliciting an immune response, e.g., a neutralizing antibody and/or cellular immune response, to treat the disease, e.g., by reducing, diminishing, abrogating, ameliorating, or eliminating the disease, and/or the symptoms thereof. In an embodiment, the immune response is a B cell response, which results in the production of antibodies, e.g., neutralizing antibodies, directed against the immunogen or immunogenic composition comprising the antigen or antigen sequence. In a manner similar to the foregoing, in some embodiments, an immunogenic composition or vaccine can be prophylactic. In some embodiments, an immunogenic composition or vaccine can be therapeutic. In an embodiment, the disease is influenza (flu).

By “immunogenic composition” is meant a composition comprising an antigen, antigen sequence, or immunogen, wherein the composition elicits an immune response in an immunized subject.

The term “immunize” (or immunization) refers to rendering a subject protected from a disease, infectious disease, or pathology, or the symptoms thereof, caused by an H3 virus, such as by immunization with an immunogenic composition.

The term “influenza virus” refers to a segmented negative-strand RNA virus that belongs to the Orthomyxoviridae family of viruses. There are three types of Influenza viruses: A, B and C. Influenza A viruses infect a wide variety of birds and mammals, including humans, horses, marine mammals, pigs, ferrets, and chickens. In animals, most influenza A viruses cause mild localized infections of the respiratory and intestinal tract. However, highly pathogenic influenza A strains, such as H3N2, cause systemic infections in poultry in which mortality may reach 100%.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 5%, 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid, protein, or peptide is purified if it is substantially free of cellular material, debris, non-relevant viral material, or culture medium when produced by recombinant DNA techniques, or of chemical precursors or other chemicals when chemically synthesized. Purity and homogeneity are typically determined using standard purification methods and analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. The term “isolated” also embraces recombinant nucleic acids, proteins or viruses, as well as chemically synthesized nucleic acids or peptides.

By “isolated polynucleotide” is meant a nucleic acid (e.g., a DNA molecule) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 40%, by weight, at least 50%, by weight, at least 60%, by weight, free from the proteins and naturally-occurring organic molecules with which it is naturally associated. Preferably, an isolated polypeptide preparation is at least 75%, more preferably at least 90%, and most preferably, at least 99%, by weight, free from the proteins and naturally-occurring organic molecules with which it is naturally associated. An isolated polypeptide may be obtained, for example, by extraction from a natural source; by expression of a recombinant nucleic acid encoding such a polypeptide; or by chemically synthesizing the protein. Purity can be measured by any standard, appropriate method, for example, column chromatography, polyacrylamide gel electrophoresis, or by HPLC analysis. An isolated polypeptide can refer to broadly active virus immunogen polypeptide generated by the methods described herein.

By “linker” is meant one or more amino acids that serve as a spacer between two polypeptides or peptides of a fusion protein.

By “marker” is meant any protein or polynucleotide having an alteration in expression level or activity that is associated with a disease, condition, pathology, or disorder.

A “Matrix (M1) protein” refers to an influenza virus structural protein found within the viral envelope. M1 is thought to function in assembly and budding of virus following infection of a cell.

The term “Neuraminidase (NA)” refers to an influenza virus membrane glycoprotein. NA is involved in the destruction of the cellular receptor for the viral HA by cleaving terminal sialic acid residues from carbohydrate moieties on the surfaces of infected cells. NA also cleaves sialic acid residues from viral proteins, preventing aggregation of viruses. NA (along with HA) is one of the two major influenza virus antigenic determinants.

As used herein, “obtaining” as in “obtaining an agent” includes synthesizing, isolating, purchasing, or otherwise acquiring the agent.

The term “operably linked” refers to nucleic acid sequences as used herein. By way of example, a first nucleic acid sequence is operably linked to a second nucleic acid sequence when the first nucleic acid sequence is placed in a functional relationship with the second nucleic acid sequence. For instance, a promoter is operably linked to a coding sequence if the promoter affects (allows) the transcription or expression of the coding sequence. Generally, operably linked DNA sequences are contiguous and, where necessary to join two protein-coding regions, are in the same reading frame.

The nucleotide sequence encoding an H3 HA protein generated by the described methods can be optimized for expression in mammalian cells via codon-optimization and RNA optimization (such as to increase RNA stability) using procedures and techniques practiced in the art.

A broadly reactive, pan-epitopic immunogen (or immunogenic antigen), such as H3 influenza hemagglutinin (HA) protein, for eliciting an immune response in a subject is produced from the methods, described herein, of generating a broadly reactive antigen derived from a pathogen, such as an influenza HA protein antigen, which possesses a collective set of strongly immunogenic epitopes (also called antigenic determinants. In the case of H3 influenza HA antigen, the methods described herein are based on focused analysis of populations of similar H3 viral HA proteins to generate a “pan-epitopic” H3 antigen that is suitable for use as an immunogen, such as a vaccine, which elicits a broadly reactive immune response, e.g., a neutralizing antibody response, against a plurality of H3 virus types which express HA proteins on the viral surface, when introduced into a host subject, in particular, a human subject infected with H3 virus. The immunogen resulting from the practice of the methods is advantageous for providing an anti-H3 virus immunogen (or a vaccine) that elicits a broadly active immune response against H3 influenza virus HA antigens with antigenic variability and similarity, and treats or protects against infection and disease caused by many different influenza virus types and subtypes.

By “open reading frame (ORF)” is meant a series of nucleotide triplets (codons) that code for amino acids without any termination codons. These sequences are usually translatable into a peptide or polypeptide.

As used herein, an influenza virus “outbreak” refers to a collection of virus isolates from within a geographical location (e.g., within a single country) in a given period of time (e.g., in a year).

The term “pharmaceutically acceptable vehicle” refers to conventional carriers (vehicles) and excipients that are physiologically and pharmaceutically acceptable for use, particularly in mammalian, e.g., human, subjects. Such pharmaceutically acceptable vehicles are known to the skilled practitioner in the pertinent art and can be readily found in Remington's Pharmaceutical Sciences, by E. W. Martin, Mack Publishing Co., Easton, Pa., 15th Edition (1975) and its updated editions, which describes compositions and formulations suitable for pharmaceutical delivery of one or more therapeutic compositions, such as one or more influenza vaccines, and additional pharmaceutical agents. In general, the nature of a pharmaceutically acceptable carrier depends on the particular mode of administration being employed. For instance, parenteral formulations usually comprise injectable fluids/liquids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers may include, for example, pharmaceutical grades of mannitol, lactose, starch, or magnesium stearate, which typically stabilize and/or increase the half-life of a composition or drug. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

By “plasmid” is meant a circular nucleic acid molecule capable of autonomous replication in a host cell.

By “polypeptide” (or protein) is meant a polymer in which the monomers comprise amino acid residues that are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “residue” or “amino acid residue” also refers to an amino acid that is incorporated into a protein, polypeptide, or peptide.

Conservative amino acid substitutions are those substitutions that, when made, least interfere with the properties of the original protein, that is, the structure and especially the function of the protein is conserved and is not significantly changed by such substitutions. Examples of conservative amino acid substitutions are known in the art, e.g., as set forth in, for example, U.S. Publication No. 2015/0030628. Conservative substitutions generally maintain (a) the structure of the polypeptide backbone in the area of the substitution, for example, as a sheet or helical conformation; (b) the charge or hydrophobicity of the molecule at the target site; and/or (c) the bulk of the side chain

The substitutions that are generally expected to produce the greatest changes in protein properties are non-conservative, for instance, changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

By “parameter” is meant a variable or condition that is assessed or taken into consideration. Examples of parameters taken into consideration in embodiments of the methods include season (time), geography, clade, species or type.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “promoter” is meant an array of nucleic acid control sequences, which direct transcription of a nucleic acid. A promoter includes necessary nucleic acid sequences near the start site of transcription. A promoter also optionally includes distal enhancer or repressor sequence elements. A “constitutive promoter” is a promoter that is continuously active and is not subject to regulation by external signals or molecules. In contrast, the activity of an “inducible promoter” is regulated by an external signal or molecule (for example, a transcription factor). By way of example, a promoter may be a CMV promoter.

As will be appreciated by the skilled practitioner in the art, the term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to routine methods, such as fractionation, chromatography, or electrophoresis, to remove various components of the initial preparation, such as proteins, cellular debris, and other components.

A “recombinant” nucleic acid, protein or virus is one that has a sequence that is not naturally occurring or that has a sequence that is made by an artificial combination of two otherwise separated segments of sequence. Such an artificial combination is often accomplished by chemical synthesis or by the artificial manipulation of isolated segments of nucleic acids, for example, by genetic engineering techniques. A “non-naturally occurring” nucleic acid, protein or virus is one that may be made via recombinant technology, artificial manipulation, or genetic or molecular biological engineering procedures and techniques, such as those commonly practiced in the art.

By “reduces” is meant a negative alteration of at least 5%, 10%, 25%, 30%, 40%, 50%, 75%, 80%, 85%, 90%, 95%, 98%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least 16 amino acids, preferably at least 20 amino acids, more preferably at least 25 amino acids, and even more preferably 35 amino acids, 50 amino acids, or 100 amino acids. For nucleic acids, the length of the reference nucleic acid sequence will generally be at least 50 nucleotides, preferably at least 60 nucleotides, more preferably at least 75 nucleotides, and even more preferably 100 nucleotides or 300 nucleotides or any integer thereabout or therebetween.

By “specifically binds” is meant a compound or antibody that recognizes and binds a polypeptide, such as a virus polypeptide, peptide, or vaccine product, but which does not substantially recognize and bind other molecules in a sample, for example, a biological sample, which naturally includes a polypeptide, such as a virus polypeptide or peptide.

Nucleic acid molecules useful in the methods described herein include any nucleic acid molecule that encodes a polypeptide as described, or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pairing to form a double-stranded molecule between complementary polynucleotide sequences (e.g., a gene), or portions thereof, under various conditions of stringency. (See, e.g., Wahl, G. M. and S. L. Berger, (1987), Methods Enzymol., 152:399; Kimmel, A. R., (1987), Methods Enzymol. 152:507).

By way of example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 m/ml denatured salmon sperm DNA (ssDNA). In a most preferred embodiment, hybridization will occur at 42° C. in 250 mM NaCl, 25 mM trisodium citrate, 1% SDS, 50% formamide, and 200 m/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, or at least 80% or 85%, or at least or equal to 90%, 95% or even 99% identical at the amino acid level or nucleic acid to the sequence used for comparison.

“Sequence identity” refers to the similarity between amino acid or nucleic acid sequences that is expressed in terms of the similarity between the sequences. Sequence identity is frequently measured in terms of percentage identity (or similarity or homology); the higher the percentage, the more similar the sequences are. Homologs or variants of a given gene or protein will possess a relatively high degree of sequence identity when aligned using standard methods. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. In addition, other programs and alignment algorithms are described in, for example, Smith and Waterman, 1981, Adv. Appl. Math. 2:482; Needleman and Wunsch, 1970, J Mol. Biol. 48:443; Pearson and Lipman, 1988, Proc. Natl. Acad. Sci. U.S.A. 85:2444; Higgins and Sharp, 1988, Gene 73:237-244; Higgins and Sharp, 1989, CABIOS 5:151-153; Corpet et al., 1988, Nucleic Acids Research 16:10881-10890; Pearson and Lipman, 1988, Proc. Natl. Acad. Sci. U.S.A. 85:2444; and Altschul et al., 1994, Nature Genet. 6:119-129. The NCBI Basic Local Alignment Search Tool (BLAST™) (Altschul et al. 1990, J. Mol. Biol. 215:403-410) is readily available from several sources, including the National Center for Biotechnology Information (NCBI, Bethesda, Md.) and on the Internet, for use in connection with the sequence analysis programs blastp, blastn, blastx, tblastn and tblastx.

By “subject” is meant an animal, e.g., a mammal, including, but not limited to, a human, a non-human primate, or a non-human mammal, such as a bovine, equine, canine, ovine, or feline mammal, or a sheep, goat, llama, camel, or a rodent (rat, mouse), gerbil, or hamster. In a nonlimiting example, a subject is one who is infected with an H3 virus, or who is at risk of infection by such virus, or who is susceptible to such infection. In particular aspects as described herein, the subject is a human subject, such as a patient.

Ranges provided herein are understood to be shorthand for all of the values within the range, inclusive of the first and last stated values. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or greater, consecutively, such as to 100 or greater.

As used herein, the terms “treat,” treating,” “treatment,” and the like refer to reducing, diminishing, decreasing, abrogating, ameliorating, or eliminating, a disease, condition, disorder, or pathology, and/or symptoms associated therewith. While not intending to be limiting, “treating” typically relates to a therapeutic intervention that occurs after a disease, condition, disorder, or pathology, and/or symptoms associated therewith, have begun to develop so as to reduce the severity of the disease, etc., and the associated signs and symptoms. It will be appreciated that, although not precluded, treating a disorder or condition does not require that the disease, condition, disorder, pathology, or the symptoms associated therewith, be completely eliminated.

As used herein, the terms “prevent,” “preventing,” “prevention,” “prophylactic treatment” and the like, refer to inhibiting or blocking a disease state, or the full development of a disease in a subject, or reducing the probability of developing a disease, disorder or condition in a subject, who does not have, but is at risk of developing, or is susceptible to developing, a disease, disorder, or condition.

As referred to herein, a “transformed” cell is a cell into which a nucleic acid molecule or polynucleotide sequence has been introduced by molecular biology techniques. As used herein, the term “transformation” encompasses all techniques by which a nucleic acid molecule or polynucleotide may be introduced into such a cell, including transfection with viral vectors, transformation with plasmid vectors, and introduction of naked nucleic acid (DNA or RNA) by electroporation, lipofection, and particle gun acceleration.

By “vaccine” is meant a preparation of immunogenic material (e.g., protein or nucleic acid; vaccine) capable of stimulating (eliciting) an immune response, administered to a subject to treat a disease, condition, or pathology, or to prevent a disease, condition, or pathology, such as an infectious disease (caused by H3 virus infection, for example). The immunogenic material may include, for example, attenuated or killed microorganisms (such as attenuated viruses), or antigenic proteins, peptides or DNA derived from such microorganisms. Vaccines may elicit a prophylactic (preventative) immune response in the subject; they may also elicit a therapeutic response immune response in a subject. As mentioned above, methods of vaccine administration vary according to the vaccine, and can include routes or means, such as inoculation (intravenous or subcutaneous injection), ingestion, inhalation, or other forms of administration. Inoculations can be delivered by any of a number of routes, including parenteral, such as intravenous, subcutaneous or intramuscular. Vaccines may also be administered with an adjuvant to boost the immune response.

As used herein, a “vector” refers to a nucleic acid (polynucleotide) molecule into which foreign nucleic acid can be inserted without disrupting the ability of the vector to replicate in and/or integrate into a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes in a host cell. In some embodiments of the present disclosure, the vector encodes an influenza HA, NA or M1 protein. In some embodiments, the vector is the pTR600 expression vector (U.S. Patent Application Publication No. 2002/0106798; Ross et al., 2000, Nat Immunol. 1(2):102-103; and Green et al., 2001, Vaccine 20:242-248).

By “virus-like particle (VLP)” is meant virus particles made up of one of more viral structural proteins, but lacking the viral genome. Because VLPs lack a viral genome, they are non-infectious and yield safer and potentially more-economical vaccines and vaccine products. In addition, VLPs can often be produced by heterologous expression and can be easily purified. Most VLPs comprise at least a viral core protein that drives budding and release of particles from a host cell. One example of such a core protein is influenza M1. In some embodiments herein, an H3 influenza VLP comprises the HA, NA and M1 proteins. As described herein, H3 influenza VLPs can be produced by transfection of host cells with plasmids encoding the H3 HA, NA and M1 proteins. After incubation of the transfected cells for an appropriate time to allow for protein expression (such as for approximately 72 hours), VLPs can be isolated from cell culture supernatants. By way of example, a protocol for purifying or isolating influenza VLPs from cell supernatants involves low speed centrifugation (to remove cell debris), vacuum filtration and ultracentrifugation of the VLPs through 20% glycerol. A virus-like particle may also include a subviral particle (SVP), which is typically smaller in size than a virus and constitutes a particle without a virus capsid or genome.

A “computer readable medium” refers to any article of manufacture that contains data that can be read by a computer (non-transitory media) or a carrier wave signal carrying data that can be read by a computer. Such computer readable media include, without limitation, magnetic media, such as a disk, tape, or cards; optical media such as CD-ROM or writeable compact disk; magneto-optical media in disk, tape, or card form; paper media, such as punched cards or paper tape; or on carrier wave signal received through a network, wireless network or modem, internet, including, but not limited to, radio-frequency signals, satellite signals, and infrared signals.

Unless specifically stated or obvious from context, as used herein, the term “or” is understood to be inclusive. Unless specifically stated or obvious from context, as used herein, the terms “a”, “an”, and “the” are understood to be singular or plural. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Unless specifically stated or obvious from context, as used herein, the term “about” is understood as within a range of normal tolerance in the art, for example within 2 standard deviations of the mean. About may be understood as being within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 depicts a schematic flowchart of the steps of the method for generating a broadly reactive antigen of H3 virus for use in eliciting a broadly active immune response, e.g., in the form of neutralizing antibodies, against H3 influenza virus strains as described herein. Sequences are first obtained from a group of viruses. The sequences are then aligned and partial sequences are removed. The remaining sequences are used to generate a phylogenetic tree, and clusters that are ≥95%-98% (particularly ≥98%) identical are extracted from the tree. These extracted sequences are then used to generate a “primary” (1°) ‘broadly reactive’ sequence with optimal alignment of identity among sequences. This process is repeated until all clusters from the phylogenetic tree have been addressed. Next all (1°) ‘broadly reactive’ sequences are consolidated and aligned with one another. A new phylogenetic tree is then generated using the 1° ‘broadly reactive’ sequences. From this tree clusters of ≥3 1° ‘broadly reactive’ sequences are extracted, and from these a “secondary”) (2°) ‘broadly reactive’ sequence is generated. This process is repeated until all of the clusters of (1°) ‘broadly reactive’ sequences have been addressed. Using the “secondary”) (2°) ‘broadly reactive’ sequences, a “backbone” ‘broadly reactive’ sequence is generated. The (2°) ‘broadly reactive’ sequences are then inserted into rolling scenarios 1, 2, and/or 3 (FIG. 2A) to generate final sequences. The final sequences are then checked for uniqueness against the sequences downloaded in step one.

FIGS. 2A-2D illustrate aspects of the methods described herein for generating non-naturally occurring, broadly reactive, pan-epitopic, H3 influenza virus antigen sequences, taking into account influenza season and geographical location, e.g., 2002 Southern Hemisphere or 2002-2003 Northern Hemisphere influenza season. FIG. 2A presents three scenarios for rolling forward new antigen sequences into a backbone sequence. Following the design of the initial backbone sequence (described here as a set of sequences derived from Years 1-3), the three different scenarios shown depict how new sequences, from subsequent years, are added to the set of sequences derived from Years 1-3. FIGS. 2B-2D present further steps of the methods as described herein. FIG. 2B depicts steps in which primary sequences are first aligned by a season, and then are aligned as secondary sequences per season. All secondary alignments are compared and aligned into a multi-seasonal, secondary aligned sequence to generate the backbone sequence as referred to in FIG. 2A. FIG. 2C presents a schematic of the phylogenetic tree analysis associated with generating a sequence in accordance with Scenario #1 of FIG. 2A. FIG. 2D presents a schematic of the phylogenetic tree analysis associated with generating sequences in accordance with Scenario #2 of FIG. 2A, in which involves extending a sequence by one season, and then another season.

FIG. 3 shows the amino acid sequences of two, representative, broadly reactive HA polypeptides of the H3 influenza A virus strain, referred to as Ross-1 and Ross-2 HA antigens (or immunogens) herein, which were generated by the methodology described herein. The HA polypeptide is composed of an HA1 head portion and an HA2 tail or stalk portion as described herein. Nucleic acid sequences encoding these polypeptides can be used to generate virus-like particles (VLPs) containing the H3 protein antigens, which are used as immunogens/vaccines to generate neutralizing antibodies in immunized subjects. As shown in FIG. 3, the full-length Ross-1 and Ross-2 HA polypeptides are 566 amino acids in length.

FIG. 4 shows hemagglutination inhibition (HA1) antibody titers against HA antigens from different strains of H3 viruses in different geographies and seasons (Nan/95, Syd/97, Pan/99, Fuj/02, NY/04, Wisc/05, Uruguay/07, Perth/09, Vic/11, Tx/12, Sz/13, HK/14). The antibodies were generated by administering to animals (mice) VLP immunogens produced using the Ross-1 and Ross-2 HA polypeptide sequences as shown in FIG. 3. The HA1 assay was performed as described in Example 3. The HA1 titers of neutralizing antibodies in the serum samples obtained from the immunized animals are presented in the table in this figure.

### DETAILED DESCRIPTION OF THE DISCLOSURE

The H3 influenza virus routinely spreads in humans and causes seasonal flu epidemics. The H3 virus typically causes severe flu disease and adapts to evade being eradicated by constantly changing its surface proteins, such as the HA protein. H3 influenza A virus was found to be a dominant strain in the U.S. and worldwide, e.g., in Australia and the United Kingdom, in the flu season that extended from the year 2017 into 2018. The H3 strain was particularly problematic to treat because of its unusually high rate of mutation and an inability to generate vaccines that were effective against the relatively rapid changes that occurred in its HA surface protein, such as during production of a vaccine against this strain.

Featured herein are methods for generating synthetic (non-naturally occurring), immunogenic antigens, e.g., protein and glycoprotein antigens, derived from the influenza (“flu”) hemagglutinin (HA) protein of the H3 strain of influenza A virus, that elicit a potent, broadly reactive and long-lasting immune response in a subject, particularly, a human subject. Such immunogenic antigens are also referred to as “immunogens,” “vaccine immunogens” or “vaccines” herein.

The methods described herein provide immunogens that protect against disease caused by the influenza H3 strain, or seasonal influenza H3 strains, spanning several years, including drifted strains not yet in existence. In an embodiment, the invention features fully synthetic protein antigens, such as influenza H3 virus HA protein antigens, generated by the methods. Such H3 HA antigens are synthetic proteins not found in nature, yet they retain all of the functions of a natural H3 HA viral protein and are immunogenic, i.e., they can elicit an immune response, in particular, a broadly active immune response in the form of neutralizing antibodies and/or reactive T lymphocytes, following administration or delivery to, or introduction into, a subject. Also provided are immunogenic compositions, e.g., vaccines, comprising the synthetic H3 virus protein antigens, or nucleic acids encoding the antigens.

In accordance with the methods described herein, an H3 HA amino acid sequence and a protein antigen having such sequence are generated for use as an immunogen, immunogenic composition, e.g., a vaccine, that elicits a broadly reactive immune response in a subject, particularly a human subject, to whom the composition is administered. The immunogenic H3 antigen or vaccine is generated from the focused, comparative analysis and clustering of many H3 HA amino acid sequences so as to afford broadly active protection against H3 flu virus proteins having sequence variability and similarity over several years. The resulting H3 virus immunogens comprise antigenic determinants that represent different “antigenic spaces” derived from the sequences of many H3 virus strains analyzed based on seasonal periods of time (either overlapping or non-overlapping seasonal time periods). By way of example, the methods can involve wild-type H3 influenza strains assessed from a defined time period, e.g., an approximately 40-year time period, such as from 1968-2013.

The assessed overlapping or non-overlapping seasonal time periods may encompass different intervals of time, for example, 5 months, 6 months, 7 months, eight months, nine months, 10 months, 11 months, 1 year, 2 years, 3 years, 4 years, 5 years, 6 years, 7 years, or 8 years, including time intervals therebetween. The overall time period over which the analysis of H3 HA sequence identities and differences are made, based on the H3 HA sequences assessed in the more defined time periods, may also encompass different lengths of time, such as an overall time period of about or equal to 10 years, about or equal to 15 years, about or equal to 20 years, about or equal to 25 years, about or equal to 30 years, about or equal to 40 years, about or equal to 50 years, and the like, including time intervals therebetween.

The methods as described herein involve the generation of seasonal pan-epitopic, broadly reactive antigens of H3 influenza virus and subtypes thereof, especially antigens containing sequences based on H3 drift variants, wherein the antigens are designed to generate a broadly active immune response, particularly in the form of neutralizing antibodies, in a subject, particularly a human subject. As described infra, Examples 1 and 2 relate to the design of a method to generate and update on a seasonal/annual basis immunogenic antigens derived from H3 influenza virus, wherein the antigens have broadly reactive antigenic determinants. Such a design is beneficial for eliciting an immune response (e.g., production of neutralizing antibodies) against the H3 virus where multiple strains of H3 co-circulate at one time. The method further provides immunogenic antigens derived from H3 virus, that frequently mutates parts of its genome to escape immune pressure, and as a consequence, evades immune surveillance in a subject whose immune system is not primed or stimulated to generate antibodies against antigenic epitopes (determinants) on the H3 antigens following infection. The methodology described herein results in the design of synthetic H3 antigens, e.g., HA antigen, having amino acid (or polynucleotide) sequences that will elicit greater numbers of neutralizing antibodies against H3 drift variants within and across multiple seasons compared with wild-type antigen sequences.

The H3 HA proteins generated through this methodology for use as a vaccine may afford protection against many H3 virus strains over several years. The broadly reactive H3 influenza proteins and vaccines described herein are advantageous in that they are designed to provide broader and longer-lasting protection against several seasonal H3 flu strains (or clades) prevalent in different geographical locations. In addition, because the methods do not rely on the annual selection of a given strain for making an immunogenic antigen or vaccine, the year-round manufacturing of an H3 flu vaccine that is broadly cross-protective against many strains or clades of H3 flu viruses is achieved. Thus, the methods described herein afford a universal and broad-spectrum H3 flu vaccine that may alleviate the need for a seasonal flu vaccine against the H3 strain and subtypes of influenza virus that is administered annually.

The immunogenic H3 virus HA antigens generated from the methods described herein may be used in immunogenic compositions (e.g., influenza vaccines) that can afford protective immunity against H3 influenza infection and disease in a subject. The protective immunity is provided in the subject through the elicitation of potent, broadly reactive, anti-H3 HA specific antibody responses that protect the subject against drifted, seasonal H3 influenza virus strains and pandemic H3 influenza virus strains. The methods provide an advantage over prior and traditional processes of manufacturing immunogenic compositions directed against H3 virus (e.g., H3 influenza vaccines), which typically depend on the selection of candidate vaccine viruses by public health authorities following analysis of data collected through active surveillance of influenza viruses circulating each year.

### Influenza Virus

Influenza viruses are segmented negative-strand RNA viruses that belong to the Orthomyxoviridae family. There are three types of Influenza viruses: A, B and C. Influenza A viruses infect a wide variety of birds and mammals, including humans, horses, marine mammals, pigs, ferrets, and chickens. In animals, most influenza A viruses cause mild localized infections of the respiratory and intestinal tract. However, highly pathogenic influenza A strains, such as H3, cause systemic infections in poultry in which mortality may reach 100%. Animals infected with influenza A often act as a reservoir for the influenza viruses and certain subtypes have been shown to cross the species barrier to humans in whom they can cause severe disease and devastating flu outbreaks that can lead to death of the infected human subjects.

Influenza A viruses can be classified into subtypes based on allelic variations in antigenic regions of two genes that encode surface glycoproteins, namely, hemagglutinin (HA) and neuraminidase (NA) which are required for viral attachment and cellular release. Currently, sixteen subtypes of HA (H1-H16) and nine NA (N1-N9) antigenic variants are known for influenza A virus. Previously, only three subtypes were known to circulate in humans (H1N1, H1N2, and H3N2). However, in recent years, for example, the pathogenic H5N1 subtype of avian influenza A has been reported to cross the species barrier and infect humans as documented in Hong Kong in 1997 and 2003, leading to the death of several patients.

In humans, the avian influenza virus infects cells of the respiratory tract as well as the intestinal tract, liver, spleen, kidneys and other organs. Symptoms of avian influenza infection include fever, respiratory difficulties, including shortness of breath and cough, lymphopenia, diarrhea and difficulties regulating blood sugar levels. In contrast to seasonal influenza, the group most at risk is healthy adults which make up the bulk of the population. Due to the high pathogenicity of certain avian influenza A subtypes, particularly H3, and their demonstrated ability to cross over to infect humans, there is a significant economic and public health risk associated with these viral strains, including a real epidemic and pandemic threat.

The influenza A virus genome encodes nine structural proteins and one nonstructural (NS1) protein with regulatory functions. The influenza virus segmented genome contains eight negative-sense RNA (nsRNA) gene segments (PB2, PB1, PA, NP, M, NS, HA and NA) that encode at least ten polypeptides, including RNA-directed RNA polymerase proteins (PB2, PB1 and PA), nucleoprotein (NP), neuraminidase (NA), hemagglutinin, e.g., subunits HA1 frequently referred to as the “head” subunit; and HA2, frequently referred to as the “tail” or “stalk” subunit; the matrix proteins (M1 and M2); and the non-structural proteins (NS1 and NS2) (See, e.g., Krug et al., 1989, In: The Influenza Viruses, R. M. Krug, ed., Plenum Press, N.Y., pp. 89 152).

The ability of influenza virus, e.g., H3, to cause widespread disease is due to its ability to evade the immune system by undergoing antigenic change, which is believed to occur when a host is infected simultaneously with both an animal influenza virus and a human influenza virus. During mutation and reassortment in the host, the virus may incorporate an HA and/or NA surface protein gene from another virus into its genome, thereby producing a new influenza subtype and evading the immune system.

Because of antigenic variation (drift) in the circulating strains of H3 influenza virus, in particular, in the HA and NA proteins of the virus, the efficacy of vaccines against H3 influenza virus has frequently been less than optimal and sub-par. The methods described herein provide broadly reactive, pan-epitopic HA or NA antigens of H3 influenza virus that generate a broadly reactive immune response, particularly, in the form of neutralizing antibodies that bind to the H3 viral antigens and neutralize the activity of the virus (e.g., its ability to infect cells), to treat H3 influenza and its symptoms more effectively.

### Influenza Virus Hemagglutinin (HA) and Neuraminidase (NA) Proteins

HA is a viral surface glycoprotein that generally comprises approximately 560 amino acids (e.g., 566 amino acids) and represents 25% of the total virus protein. As described herein, HA is a protein antigen that is highly useful as an immunogen against the H3 virus because it contains a diverse repertoire of epitopes against which antibodies are generated in a subject or host that encounters the H3 HA antigen during infection.

HA is responsible for adhesion of the viral particle to, and its penetration into, a host cell, particularly, in the respiratory epithelium, in the early stages of infection. Cleavage of the virus HA0 precursor into the HA1 and HA2 sub-fragments is a necessary step in order for the virus to infect a cell. Thus, cleavage is required in order to convert new virus particles in a host cell into virions capable of infecting new cells. Cleavage is known to occur during transport of the integral HA0 membrane protein from the endoplasmic reticulum of the infected cell to the plasma membrane. In the course of transport, HA undergoes a series of co- and post-translational modifications, including proteolytic cleavage of the precursor HA into the amino-terminal fragment HA1 (“head”) and the carboxy terminal HA2 (“tail” or “stalk”). One of the primary difficulties in growing H3 influenza strains in primary tissue culture or established cell lines arises from the requirement for proteolytic cleavage activation of the influenza hemagglutinin in the host cell.

Although it is known that an uncleaved HA can mediate attachment of the virus to its neuraminic acid-containing receptors on a cell surface, it cannot perform the next step in the infectious cycle, which is fusion. It has been reported that exposure of the hydrophobic amino terminus of HA2 by cleavage is required so that it can be inserted into the target cell, thereby forming a bridge between the virus and the target cell membranes. This process is followed by fusion of the two membranes and entry of the virus into the target cell.

Proteolytic activation of HA involves cleavage at an arginine residue by a trypsin-like endoprotease, which is often an intracellular enzyme that is calcium-dependent and has a neutral pH optimum. Since the activating proteases are cellular enzymes, the infected cell type determines whether the HA is cleaved. The HA of the mammalian influenza viruses and the nonpathogenic avian influenza viruses are susceptible to proteolytic cleavage only in a restricted number of cell types. There are also differences in host range resulting from differences in hemagglutinin cleavability which are correlated with the pathogenic properties of the virus.

Neuraminidase (NA) is a second membrane glycoprotein of the influenza viruses. The presence of viral NA has been shown to be important for generating a multi-faceted protective immune response against an infecting virus. For most influenza A viruses, NA is 413 amino acid in length, and is encoded by a gene of 1413 nucleotides. Nine different NA subtypes have been identified in influenza viruses (N1, N2, N3, N4, N5, N6, N7, N8 and N9), all of which have been found among wild birds. NA is involved in the destruction of the cellular receptor for the viral HA by cleaving terminal neuraminic acid (also called sialic acid) residues from carbohydrate moieties on the surfaces of infected cells. NA also cleaves sialic acid residues from viral proteins, preventing aggregation of viruses. Using this mechanism, it is hypothesized that NA facilitates the release of viral progeny by preventing newly formed viral particles from accumulating along the cell membrane, as well as by promoting transportation of the virus through the mucus present on the mucosal surface. NA is an important antigenic determinant that is subject to antigenic variation.

In addition to the surface proteins HA and NA, H3 influenza virus comprises six additional internal genes, which give rise to eight different proteins, including polymerase genes PB1, PB2 and PA, matrix proteins M1 and M2, nucleoprotein (NP), and non-structural proteins NS1 and NS2 (See, e.g., Horimoto et al., 2001, Clin Microbiol Rev. 14(1):129-149).

In order to be packaged into progeny virions, H3 viral RNA is transported from the nucleus as a ribonucleoprotein (RNP) complex composed of the three influenza virus polymerase proteins, the nucleoprotein (NP), and the viral RNA, in association with the influenza virus matrix 1 (M1) protein and nuclear export protein (Marsh et al., 2008, J Virol, 82:2295-2304). The M1 protein that lies within the envelope is thought to function in assembly and budding. A limited number of M2 proteins are integrated into the virions (Zebedee, 1988, J. Virol. 62:2762-2772). These M2 proteins form tetramers having H+ ion channel activity, and when activated by the low pH in endosomes, acidify the inside of the virion, thus facilitating its uncoating (Pinto et al., 1992, Cell 69:517-528). Amantadine is an anti-influenza drug that prevents viral infection by interfering with M2 ion channel activity, thus inhibiting virus uncoating.

NS1, a nonstructural protein, has multiple functions, including regulation of splicing and nuclear export of cellular mRNAs as well as stimulation of translation. The major function of NS1 seems to be to counteract the interferon activity of the host, since an NS1 knockout virus was viable, although it grew less efficiently than the parent virus in interferon-nondefective cells (Garcia-Sastre, 1998, Virology 252:324-330).

The NS2 nonstructural protein has been detected in virus particles (Richardson et al., 1991, Arch. Virol. 116:69-80; Yasuda et al., 1993, Virology 196:249-255). The average number of NS2 proteins in a virus particle was estimated to be 130-200 molecules. An in vitro binding assay has demonstrated direct protein-protein contact between M1 and NS2. NS2-M1 complexes have also been detected by immunoprecipitation in virus-infected cell lysates. The NS2 protein is thought to play a role in the export of the RNP from the nucleus through interaction with M1 protein (Ward et al., 1995, Arch. Virol. 140:2067-2073).

### Influenza Proteins and Virus-Like Particles (VLPs)

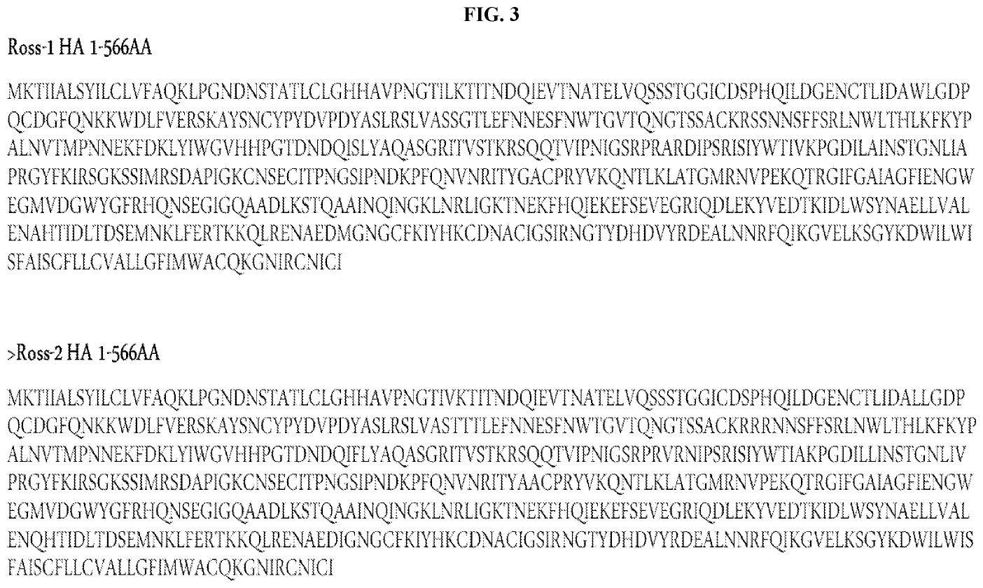

Provided by the described methods are non-naturally occurring, broadly reactive, pan-epitopic H3 influenza HA immunogenic polypeptides (immunogens) and influenza virus-like particles (VLPs) comprising an H3 HA immunogen containing diverse epitopes (antigenic determinants) that endow the HA antigen with the ability to generate a broadly active immune response against influenza and its symptoms, either prophylactic or therapeutic, following administration and delivery to a susceptible subject. By way of example, representative H3 HA immunogenic antigen sequences generated by the practice of methods described herein are presented in FIG. 3. In particular examples, the broadly reactive, pan-epitopic H3 HA polypeptides are administered as part of a VLP.

It will be understood that the H3 influenza virus immunogens and sequences described and provided herein are non-naturally occurring, broadly reactive and pan-epitopic, whether or not these characteristics and features are explicitly stated. It will also be appreciated that the H3 antigen proteins, e.g., HA, HA1 or HA2, as described herein and used as immunogens are non-naturally occurring or synthetic antigens that elicit an immune response, e.g., neutralizing antibodies, in a subject.

The H3 influenza VLPs include the viral HA proteins. In embodiments, the VLPs may include the HA1 and/or the HA2 proteins. It will be appreciated that in some cases, H3 influenza virus VLPs may include the viral NA and M1 proteins. The production of influenza VLPs has been described in the art and is within the skill and expertise of one of ordinary skill in the art. Briefly, and as described, influenza VLPs can be produced by transfection of host cells with one or more plasmids containing polynucleotide sequences that encode the HA proteins. After incubation of the transfected cells for an appropriate time to allow for protein expression (such as for approximately 72 hours), H3 VLPs can be isolated from cell culture supernatants. H3 influenza VLPs can be purified from cell supernatants using procedures practiced in the art, for example, VLPs can isolated by low speed centrifugation (to remove cell debris), vacuum filtration and ultracentrifugation through 20% glycerol.

The influenza VLPs can be used as influenza immunogens (e.g., vaccines) to elicit an immune response against H3 influenza viruses. In particular, the component, broadly reactive, pan-epitopic H3 influenza HA polypeptides of the immunogens (e.g., vaccines or VLPs) contain antigenic (pan-epitopic) determinants that are broadly reactive and serve to elicit an immune response in a subject (e.g., the production of neutralizing antibodies and/or activated T-cells) that can treat an H3 virus-infected subject (e.g., neutralize the infecting virus) and/or protect a subject against full-blown virus infection or the signs and symptoms thereof

Methods of Generating Non-Naturally Occurring, Pathogen-Derived, Immunogenic 113 Antigens which Elicit a Broadly Active Immune Response in a Subject

Described herein are improved methods for generating a non-naturally occurring, broadly reactive and immunogenic antigen of H3 influenza, such as H3 HA antigen, in which the sequence of the antigen contains a diverse repertoire of epitopic determinants that can reflect antigenic drift and sequence variability in the H3 virus's antigenic proteins, for example, over seasons (time) and in different geographic locations. In particular, the methods described herein are used to generate an H3 HA viral antigen, having an amino acid sequence that contains antigenic determinants (epitopes) derived from sequence diverse virus strains, including drift variants, against which broadly reactive neutralizing antibodies can be raised, especially when the antigen is used as an immunogenic product, (an immunogen), e.g., an antiviral vaccine, that is introduced into a subject. The methods described herein take into account both antigen sequence identities and variabilities in clustering sequences to produce secondary and tertiary sequences, as well as build on sequence identity and diversity over seasons and geography to improve immunogenicity, in generating broadly reactive and pan-epitopic H3 immunogens, thus providing a next stage technology, relative to the approach described in U.S. Pat. Nos. 8,883,171, 9,212,207, 9,309,290, 9,555,095, 9,566,327, 9,566,328 and 9,580,475.

The methods described herein thus provide a next generation technology for generating non-naturally occurring, immunogenic antigen sequences derived from H3 influenza virus. For H3, the surface proteins hemagglutinin (HA), which is responsible for binding and entry into host epithelial cells is a relevant antigen for providing a highly immunogenic sequence. The antigenic determinants or sites recognized on the H3 hemagglutinin protein by host immune systems are under constant selective pressure. Antigenic drift allows for evasion of these host immune systems by small mutations in the hemagglutinin gene that make the protein unrecognizable to pre-existing host immunity. In particular, antigenic drift and the resulting drift variants involve a continuous process of genetic and antigenic change among flu strains.

It will be understood that the H3 antigen proteins, e.g., HA, HA1 HA2, or NA, generated by the methods described herein and used as immunogens are non-naturally occurring or synthetic antigens that elicit an immune response, e.g., neutralizing antibodies, in a subject.

The methods described herein include the evaluation of relevant parameters for analyzing and generating a composite H3 viral antigen sequence that comprises epitopes reflecting sequence variability among many H3 virus subtypes, and involve a more comprehensive analysis and repeated clustering of different populations of antigen sequences of H3 viruses present in different seasons or of different virus clades or types. The methods described herein achieve a composite H3 viral antigen amino acid sequence that includes epitopic determinants ultimately derivable from both past and more recent seasons of virus infection or disease, and/or from viruses in different geographical locales, and/or from H3 viruses in different clades or types, i.e., a “pan-epitopic” antigen that elicits a broadly reactive immune response when used as an immunogen.

The present methods provide the generation of a non-naturally occurring and pan-epitopic H3 viral antigen that can elicit a broad immunogenic response when used as an immunogenic antigen, such as a vaccine (or as a component of a virus-like particle), and can involve the assessment of one or more of the following parameters: seasonality, (i.e., sequences of H3 virus HA antigen from past seasons and more recent seasons are assessed); geographical location, (i.e., sequences of H3 virus HA antigen from H3 viruses in different geographic areas or regions of the world where H3 virus outbreaks and infection have occurred are assessed); or different clades or subtypes (i.e., sequences of H3 virus HA antigens derived from H3 viruses of different clades, types, or subtypes are assessed).

In general, the methods described herein provide a non-naturally occurring, broadly reactive, pan-epitopic H3 virus-derived antigen sequence that is enhanced in the repertoire of epitopes that it contains. By way of example, an H3 HA protein antigen generated by the methods is ultimately derived from a vast number of H3 sequences that are clustered and ordered based on their sequence identity, degree of genetic relatedness and degree of genetic variability, so as to arrive at a final protein sequence, following successive (iterative) rounds of sequence clustering and sequence identity analysis, that includes a greater number of diverse antigenic determinants or epitopes per single protein antigen generated as a result of the methodology, for example, when compared with previous approaches or techniques. The H3 HA antigen sequences of immunogens generated by the methods can encompass epitopes that result from antigenic changes in the sequences of H3 HA surface antigens that arise from point mutations during viral replication, giving rise to new H3 influenza variants. As a result, the administration to a subject of an H3 immunogen generated by the methods described herein can elicit an immune response in the subject that is directed against epitopes reflecting such antigenic changes.

A broadly reactive H3 HA antigen obtained by the described methods and used as an immunogen or immunogenic composition, such as a vaccine, elicits a broadly reactive immune response in an immunocompetent subject. Thus, the present methodology of generating a pan-epitopic and immunogenic H3 HA protein antigen provides a superior vaccine that captures the antigenic epitopes of many different H3 influenza isolates, against which broadly active immune responses (e.g., broadly active neutralizing antibodies) are generated. It is noted that the terms “broadly active” and “broadly reactive” are used synonymously herein.

In an embodiment, a method of generating an antigen (e.g., a glycoprotein antigen) of H3 influenza virus is provided, in which the amino acid sequence of the H3 antigen comprises a composite sequence representing high proportion of the major and minor epitopes/antigenic determinants derived from the assessment of many amino acid sequences of the antigen by clustering the sequences based on sequence identities and differences, and selecting the most representative antigen sequences of H3, taking into account different time periods (seasons in which disease or infection caused by the H3 are prevalent), e.g., linear time periods, and geographic areas (where disease or infection caused by H3 are prevalent). Such a pan-epitopic antigen serves as a potent immunogen and comprises an H3 vaccine that is recognized and bound by broadly active neutralizing antibodies in a subject exposed to H3 virus.

Methods of generating an H3 antigen sequence and expressed immunogenic H3 antigen (e.g., a polypeptide, peptide, or a polynucleotide antigen) are described herein. The H3 antigen generated by the methods elicits an immune response after introduction into a subject, in particular, an immune response in which broadly reactive neutralizing antibodies directed against the H3 antigen are produced. In an embodiment, the antigen is a polypeptide or peptide antigen of H3 virus which currently causes disease or infection and its symptoms, such as seasonal H3 influenza, and which is native to certain geographical locales. In another embodiment, the H3 antigen is a polypeptide or peptide antigen which will, in future, cause disease and symptoms of H3 influenza. In an embodiment, the H3 antigen is a polynucleotide sequence. By way of example, representative H3 HA broadly reactive antigens generated by the methods described herein are shown in FIG. 3.

The method of generating a broadly reactive, non-naturally occurring, pan-epitopic antigen, (e.g., HA, HA1 or HA2 antigens), of an H3 virus and viruses similar thereto involves the consideration of the parameters of antigen sequences of H3 virus from a linear time range (e.g., spanning one or more flu seasons) and geography (e.g., Northern and/or Southern Hemisphere).

In a particular embodiment, a method of generating a non-naturally occurring pan-epitopic immunogen capable of generating an immune response against present and future H3 Influenza (H3) strains in a subject is provided, in which the method comprises (a) generating a phylogenetic tree comprising full length related antigen sequences derived from H3 strains present during two or more consecutive flu seasons in the Northern and Southern Hemispheres; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; and (f) generating a phylogenetic tree comprising the secondary sequences, wherein branches of the tree are combined if the substitution rate per amino acid site distance is less than about 0.001, thereby generating a non-naturally occurring pan-epitopic immunogen sequence. In an embodiment of the method, each cluster of antigen sequences in step (b) has at least 95%-98% sequence identity.

In an embodiment of the method, step (d) and step (e) are optional. In an embodiment of the method, steps (a)-(c) are repeated two or more times. In an embodiment of the method, steps (b) and (c) are repeated two or more times prior to step (d). In an embodiment of the method, steps (e) and (f) are repeated two or more times. In another embodiment of the method, steps (a) through (e) are repeated two or more times prior to performing step (f).

In an embodiment of the method, three or more clusters of antigen sequences are identified in step (b). In a particular embodiment of the method, 5 or 15 clusters of antigen sequences are identified in step (b).

In another embodiment of the method, the results or output generated by the practice of the method, such as primary, secondary and tertiary sequences, sequence clusters, sequence alignments, phylogenetic trees, or any combinations thereof, are displayed in a visual form, such as displaying the results or output on a display device. In embodiments, the method comprises displaying the generation of clusters of H3 antigen sequences in a visual form, such as displaying on a display device. In other embodiments, the method comprises displaying the generation of a phylogenetic tree comprising antigen sequences, such as the primary or secondary antigen sequences, in a visual form, such as displaying on a display device. In embodiments, the display device is a desktop computer, a laptop computer, a hand-held computer, a smart phone, a cellular telephone, a tablet computer, or a personal digital assistant.

In another embodiment of the method, the H3 immunogen sequence generated by the practice of the method is expressed in a cell as a polypeptide, protein, or peptide. In an embodiment of the method, the immunogen generated by the practice of the method is isolated and/or purified. In an embodiment of the method, the immunogen is formulated for administration to a subject in need. In an embodiment of the method, the immunogen is administered to a subject in need thereof in an effective amount to elicit an immune response in the subject. In an embodiment of the described method, the immune response elicits neutralizing antibodies. In an embodiment, the immune response is prophylactic or therapeutic.

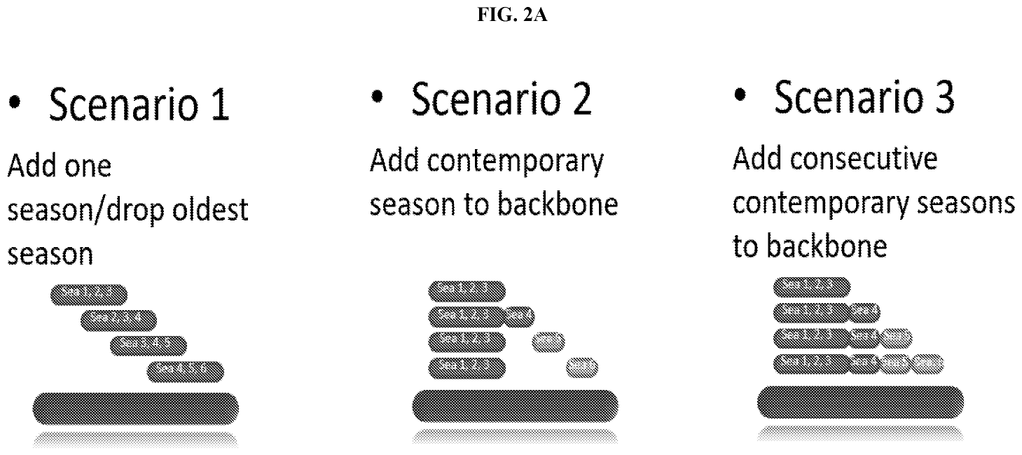

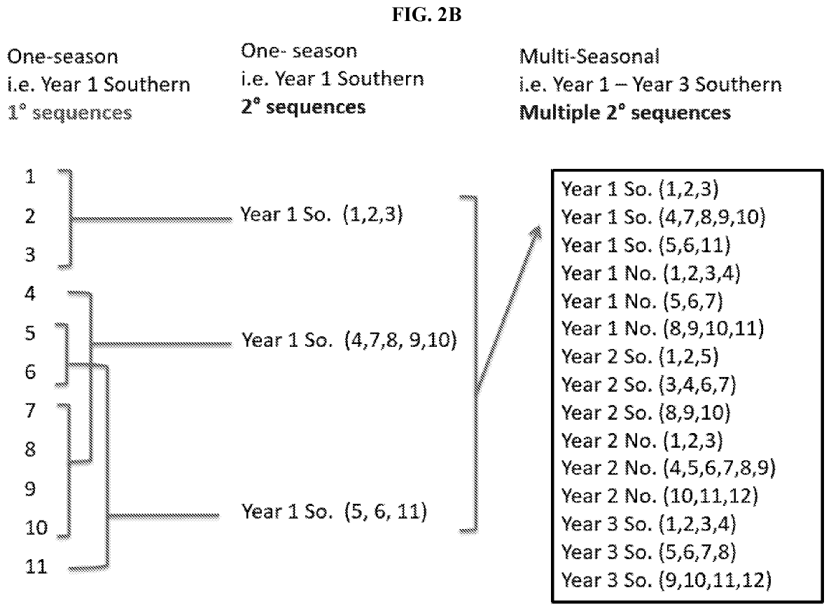

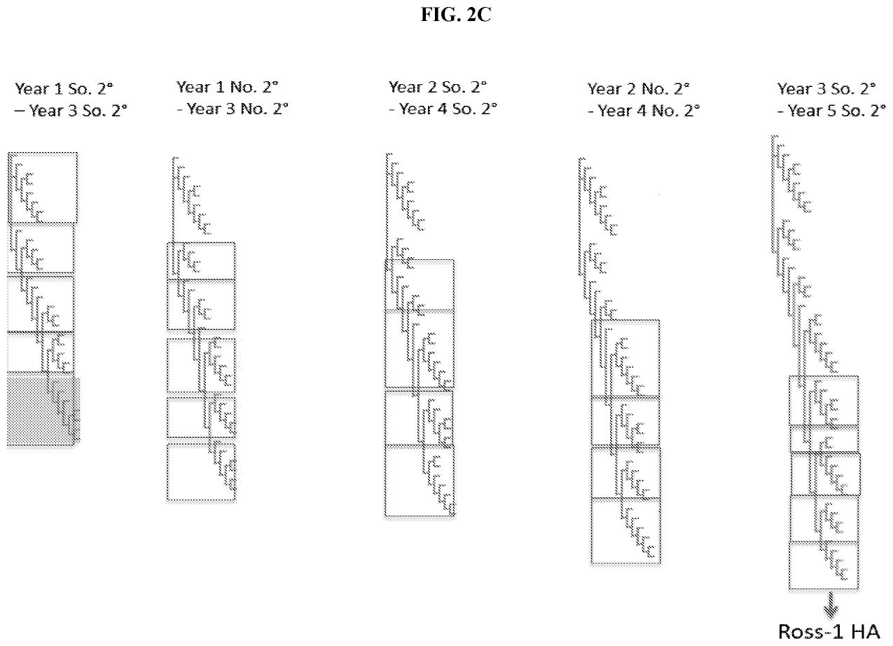

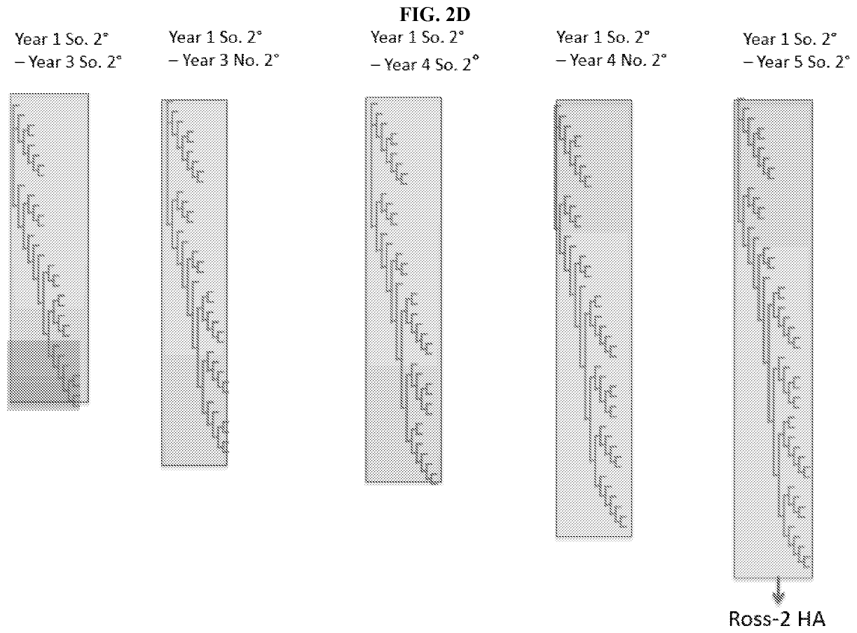

In other embodiments, the method may further involve a process to assess and include HA sequences from H3 influenza and related strains in different geographical locations (e.g., Northern or Southern Hemisphere) collected over a given time period (e.g., spans of years, such as, without limitation, 3, 4, 5, 6, 7, 8 years, or longer, time spans, for example, as indicated in Step L of the method described in Example 1 herein. The further analysis of protein sequences over time spans and seasons, and the inclusion of H3 antigen sequence variability in the analysis, provide a “rolling forward” or “seasonal rolling forward” effect, which augments the steps of the broadly reactive, H3 antigen production method, described herein, and captures past and present H3 antigen sequence similarity and variability. (FIG. 2A). Thus, in an aspect of the method, H3 HA protein sequences, for example, are included from past and present H3 viruses having diversity in their HA sequences that are assessed, for example, over temporal spans, such as seasons of infection, in different geographic locations, thus allowing the inclusion of H3 Ha antigenic determinants that can result from drift variants and can capture rare or drifted H3 antigenic determinants or epitopes within the resulting “pan-epitopic” antigen sequence generated by the method.

In one approach to a seasonal rolling methodology (Scenario 1), (FIGS. 2A-2C), sequence diversity is analyzed via phylogenetic analysis. By way of example, the process involves analyzing H3 HA1 sequences between 20-300 amino acids in length, taking into account, for example, Southern Hemisphere season: May 1 ______-September 30 ______; Northern Hemisphere season: October 1 ______-April 30 ______; aligning the sequences; constructing Jukes-Cantor NJ phylogenetic tree and assigning clusters that contain 98-99% sequence identity such that each cluster yields a primary (1°) sequence, in which 12-20 clusters for each season are optimal; aligning the 1° sequences and constructing a tree to assess sequence similarity between clusters, in which identical 1° sequences are grouped together to make a single 2° sequence, wherein a minimum of 4-8 2° sequences is optimal; aligning the 2° sequences from multiple seasons (e.g., 5 seasons) into a phylogenetic tree and dividing the sequences in the tree into clusters, with a minimum of 3-2 2° sequences; and obtaining the sequences with similarity and/or identity to yield a tertiary (3°) sequence. (FIGS. 2B and 2C; Examples 1 and 2). It will be understood that the steps of the method, and/or the parameters and/or outcomes of the steps of the method, such as sequence clusters, phylogenetic trees, primary, secondary and tertiary sequence alignments generated during the practice of the method, may be displayed in a visual form, such as on a display device.

In some embodiments of any of the foregoing aspects, the method further includes (i) reverse translating the antigen sequence (e.g., virus protein antigen) generated by the method to generate a coding sequence; and (ii) optimizing the coding sequence for expression in mammalian cells.

In another embodiment of any of the foregoing methods, the immune response elicits neutralizing antibodies. In another embodiment of any of the foregoing methods, the immune response is prophylactic or therapeutic.

In embodiments of the foregoing method, each identified cluster of H3 antigen (amino acid) sequences within the tree has at least or equal to 93%, at least or equal to 94%, at least or equal to 95%, at least or equal to 96%, at least or equal to 97%, at least or equal to 98%, or at least or equal to 99% sequence identity and at least about 0.001 substitution per site relative to the other sequences within the cluster. In other embodiments, the sequence identity range is from about or equal to 94% to about or equal to 99%, or from about or equal to 95% to about or equal to 98%, or from about or equal to 96% to about or equal to 98%.

Display of Results and Output, and/or Parameters, of Method Steps in a Visual Form, Such as on a Display Device

In an aspect related to the described methods, a non-transitory computer readable medium containing program instructions executable by a processor is provided, in which the computer readable medium contains program instructions that provide criteria that relate one or more parameters to each other, the parameters including one or more selected from the group consisting of: sequence gathering parameters, sequence alignment parameters, computationally-derived parameters of clustered sequence output and phylogenetic tree generation (related to type/subtype of pathogen, sequence identity and variability); and program instructions that input parameters of the method steps into given criteria and relate observations for one or more acquired parameters; and program instructions that converge the given criteria so as to provide an output representative of sequences containing both similarity and variability, so as to represent broadly reactive epitopes/antigenic determinants.

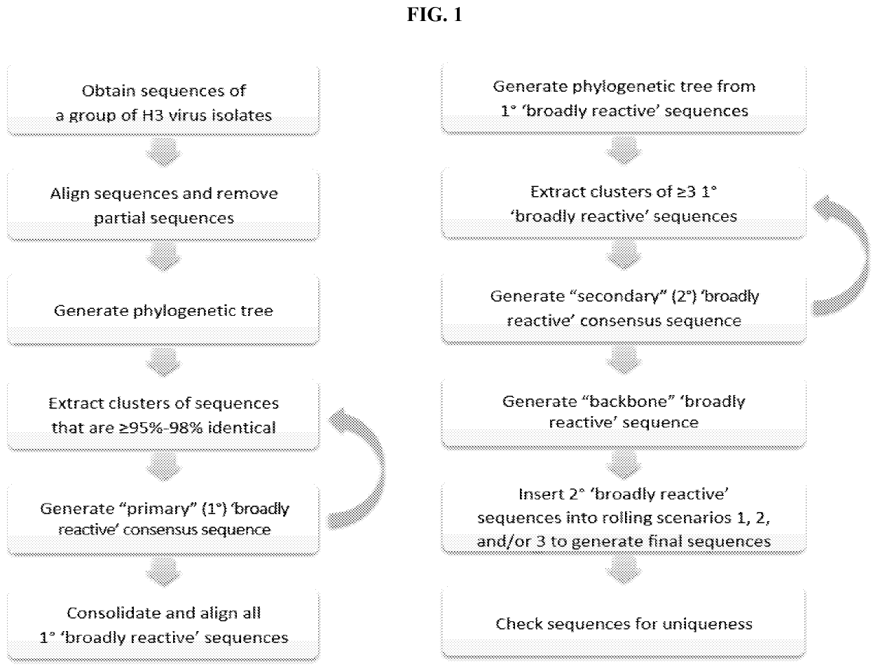

As described herein, FIG. 1 depicts a schematic flow diagram of the described method for generating non-naturally occurring, broadly reactive, pan-epitopic antigen sequences derived from a pathogen such as a virus, e.g., influenza virus of different types and subtypes. In an aspect, the method continues with displaying the input, output, and results of inputted data in one or more, or all, of the steps of the method so that one can visualize antigen sequence relationships (sequence identity and sequence variability), clustering of antigen sequences from pathogens (e.g., different clades, types, or subtypes) present in different time spans or periods (e.g., seasons) and different geographies (e.g., Northern or Southern Hemisphere). The flow chart also illustrates the structure of the logic of the different methods, which can be embodied in computer program software for execution on a computer, digital processor, or microprocessor. Those skilled in the art will appreciate that the flow chart illustrates the structure of the computer program code elements, including logic circuits on an integrated circuit that function according to the described methods. As such, one or more, or all, of the steps of the method may be practiced, with human input and involvement, by a machine component that renders the program code elements in a form that instructs a digital processing apparatus (e.g., computer) to perform a sequence of function step(s) corresponding to those shown in the flow diagram.

Accordingly, the methods as described herein are suitable for use in combination with any of a number of computer systems as are known to those skilled in the art or hereinafter developed. Such a computer system typically includes a computer, a display, and one or more input device(s). The display is any of a number of devices known to those skilled in the art for displaying images responsive to output signals from the computer, including but not limited to cathode ray tubes (CRT), liquid crystal displays (LCDS), plasma screens and the like. The signals being outputted from the computer can originate from any of a number of devices including PCI or AGP video boards or cards mounted with the housing of the computer that are operably coupled to the computer's microprocessor and the display.

The one or more input device(s) are any of a number of devices known to those skilled in the art that can be used to provide input signals to the computer for control of applications programs and other programs such as the operating system (OS) being executed within the computer. Illustratively, the input device may comprise a switch, a slide, a mouse, a track ball, a glide point or a joystick, or other such device (e.g., a keyboard having an integrally mounted glide point or mouse) by which a user can input control signals other than by means of a keyboard.

The computer typically includes a central processing unit (CPU) including one or more microprocessors such as those manufactured by Intel or AMD, Motorola or the like, random access memory (RAM), mechanisms and structures for performing I/O operations, a storage medium such as a magnetic hard disk drive(s) or other drives (fixed or removable) for storage of data, operating systems or the applications or software programs associated with the methods, including an applications program and a device for reading from and/or writing to a removable computer readable medium, such as, for example, an optical disk reader capable of reading CDROM, DVD or optical disks and readers of other types of nonvolatile memory, such as flash drives, jump drives, or spin memory that embody one or more types of non-volatile memory or storage devices.

Such a hard disk drive serves to boot or store the operating system, other applications, or systems that are to be executed on the computer, paging and swapping between the hard disk and the RAM and the like. Such data also can be stored in a removable computer readable medium such as a CD or DVD type of medium that is inserted into a device for reading and/or writing to the removable computer readable media. Alternatively, such a computer system also includes a network based computer system that includes a server, an external storage device and a network infrastructure that operably couples a plurality or more of client computer systems to the server.

The server is any of a number of servers known to those skilled in the art that are intended to be operably connected to a network so as to operably link a plurality of client computers via the network to the server and thus also to the external storage device. Such a server typically includes a central processing unit including one or more microprocessors such as those manufactured by Intel or AMD, random access memory (RAM), mechanisms and structures for performing I/O operations, a storage medium such as a magnetic hard disk drive(s), and an operating system for execution on the central processing unit.

Devices suitable for the display of data generated by the described methods include desktop computers, laptop computers, hand held computer devices, smart phone, cellular telephone, tablet computer, or personal digital assistant. Such devices typically have an OS capable of running application software (e.g., Apps) and also typically provide for wireless connection to the Internet (e.g., WI-FI, Bluetooth).

### Immunogenic 113 Antigens Obtained Through the Practice of the Method

The practice of the described method generates a H3 virus-derived, pan-epitopic antigen sequence (antigen) which is a non-naturally occurring immunogen that elicits a broadly reactive immune response in a subject following introduction, administration, or delivery of the immunogen to the subject. The route of introduction, administration, or delivery is not limited and may include, for example, intravenous, subcutaneous, intramuscular, oral, etc. routes. In an embodiment, a vaccine comprising the non-naturally occurring H3 immunogen generated by the practice of the method is provided. The vaccine may be therapeutic (e.g., administered to a subject following a symptom of disease (flu) caused by H3 virus or prophylactic (protective), (e.g., administered to a subject prior to the subject having or expressing a symptom of disease (flu), or full-blown disease, caused by H3 virus).

In an embodiment, the final amino acid sequence of the antigen, e.g., HA, arrived at through the practice of the method is reverse translated and optimized for expression in mammalian cells. As will be appreciated by the skilled practitioner in the art, optimization of the nucleic acid sequence includes optimization of the codons for expression of a sequence in mammalian cells and RNA optimization (such as RNA stability).

In an embodiment, an isolated nucleic acid molecule (polynucleotide) comprising a nucleotide sequence encoding a polypeptide or peptide antigen, such as an H3 influenza HA polypeptide (or HA1 or HA2 polypeptide), generated by the described methods is provided. In certain embodiments, the nucleotide sequence encoding the H3 HA polypeptide is at least 94%, at least 95%, at least 96%, at least 97%, at least 98% or at least 99% identical to a polynucleotide encoding an HA polypeptide (or HA1 or HA2 polypeptide) sequence shown in FIG. 3.

In other embodiments, the nucleotide sequence encoding the H3 influenza HA polypeptide (or HA1 or HA2 polypeptide) that is at least 90%, at least 91%, at least 92%, at least 93%, at least 94%, at least 95%, at least 96%, at least 97%, at least 98%, or at least 99% identical to a polynucleotide encoding an H3 HA polypeptide (or HA1 or HA2 polypeptide) sequence shown in FIG. 3 lacks the start codon encoding an N-terminal methionine.

Vectors containing a nucleotide sequence encoding a non-naturally occurring, broadly reactive polypeptide or peptide antigen, such as an H3 influenza HA polypeptide, (or HA1 or HA2 polypeptide), obtained by the described methods are provided. In some embodiments, the vectors comprise a nucleotide sequence encoding the polypeptide or peptide antigen, such as an influenza H3 HA polypeptide antigen, that is at least 94%, at least 95%, at least 96%, at least 97%, at least 98% or at least 99% identical to a polynucleotide encoding an H3 HA polypeptide (or HA1 or HA2 polypeptide) sequence shown in FIG. 3. In some embodiments, the vector further includes a promoter operably linked to the nucleotide sequence encoding the H3 HA polypeptide (or HA1 or HA2 polypeptide). In a particular embodiment, the promoter is a cytomegalovirus (CMV) promoter. In some embodiments, the nucleotide sequence of the vector is at least 85%, at least 90%, at least 95%, at least 98% or at least 99% identical to a polynucleotide encoding an H3 HA polypeptide (or HA1 or HA2 polypeptide) sequence shown in FIG. 3. In particular embodiments, the nucleotide sequence of the vector comprises the polynucleotide encoding an H3 HA polypeptide (or HA1 or HA2 polypeptide) sequence shown in FIG. 3. In embodiments, the vector is a prokaryotic or eukaryotic vector. In an embodiment, the vector is an expression vector, such as a eukaryotic (e.g., mammalian) expression vector. In another embodiment, the vector is a plasmid (prokaryotic or bacterial) vector. In another embodiment, the vector is a viral vector.

The vectors used to express an H3 virus antigen generated by the described methods, e.g., H3 viral proteins, such as the HA protein, may be any suitable expression vectors known and used in the art. The vectors can be, for example, mammalian expression vectors or viral vectors. In some embodiments, the vector is the pTR600 expression vector (U.S. Patent Application Publication No. 2002/0106798, herein incorporated by reference; Ross et al., 2000, Nat Immunol. 1(2):102-103; and Green et al., 2001, Vaccine 20:242-248).

Provided are H3 influenza virus-derived, non-naturally occurring polypeptide antigens, e.g., H3 influenza HA polypeptide antigens, or HA1 or HA2 polypeptide antigens, obtained by the described methods and produced by transfecting a host cell with an expression vector as known and used in the art under conditions sufficient to allow for expression of the polypeptide, e.g., an H3 HA, HA1 or HA2 polypeptide, in the cell. Isolated cells containing the vectors are also provided.

Also provided are non-naturally occurring, broadly reactive, pan-epitopic H3 antigen polypeptides generated by the methods described herein, such as pan-epitopic, broadly reactive H3 influenza HA polypeptides. In certain embodiments, the amino acid sequence of the polypeptide is at least 95% to 99% (inclusive) identical to the amino acid sequence of an HA, HA1 or HA2 polypeptide produced by the described methods and shown in FIG. 3. In particular embodiments, the amino acid sequence of the H3 influenza HA, HA1 or HA2 polypeptide that is at least 95% to 99% (inclusive) identical to the amino acid sequence of an HA, HA1 or HA2 polypeptide shown in FIG. 3 lacks the N-terminal methionine residue. In a particular embodiment, the amino acid sequence of the H3 influenza HA polypeptide is at least 95% to 99% (inclusive) identical to amino acids 1-566 of the H3 HA polypeptides shown in FIG. 3.

In some embodiments, fusion proteins comprising the broadly reactive, pan-epitopic H3 virus antigen polypeptides generated by the methods described herein, e.g., without limitation, the H3 influenza HA polypeptides disclosed herein, are also provided. In some embodiments, the H3 influenza HA polypeptide can be fused to any heterologous amino acid sequence to form the fusion protein. By way of example, HA1 and HA2 polypeptides may be generated independently and then fused together to produce an H3 HA polypeptide antigen, e.g., comprising 566 amino acids.

Also provided are virus-like particles (VLPs), in particular, H3 influenza VLPs, containing a pan-epitopic, broadly reactive protein antigen, e.g., H3 influenza HA, HA1 or HA2 protein, as described herein. In certain embodiments, the HA protein of the VLP is at least or equal to 94%, at least or equal to 95%, at least or equal to 96%, at least or equal to 97%, at least or equal to 98%, at least or equal to 99% or 100% identical to the H3 HA proteins as shown in FIG. 3. The virus or influenza VLPs can further include any additional viral or influenza proteins necessary to form the virus particle. In certain embodiments, the virus or influenza VLPs further include influenza neuraminidase (NA) protein, influenza matrix (M1) protein, or both.

Also provided is an H3 influenza VLP containing an H3 influenza HA, HA1 or HA2 polypeptide as described herein, produced by transfecting a host cell with a vector encoding the H3 HA, HA1 or HA2 polypeptide. Also provided in an embodiment is an H3 influenza VLP containing an H3 influenza HA polypeptide, or HA1 or HA2 polypeptide, as described herein, produced by transfecting a host cell with a vector encoding the H3 HA, HA1 or HA2 polypeptide, a vector encoding an influenza NA protein and a vector encoding an influenza M1 protein, under conditions sufficient to allow for expression of the H3 HA, NA and M1 proteins.

Collections of plasmids (vectors) are also contemplated. In certain embodiments, the collection of plasmids includes a plasmid encoding an influenza H3 NA, a plasmid encoding an H3 influenza MA, and a plasmid encoding a pan-epitopic, broadly reactive H3 HA, HA1 or HA2 protein produced by the methods as described herein. In some embodiments, the nucleotide sequence encoding a codon-optimized H3 influenza HA, HA1 or HA2 of the HA-encoding plasmid is at least 94%, at least 95%, at least 96%, at least 97%, at least 98% or at least 99% identical to a polynucleotide encoding an H3 HA amino acid sequence as shown in FIG. 3.

### Methods of Producing a Pan-Epitopic, Broadly Reactive H3 Virus-Derived Protein Sequence

In the context of the present disclosure, “broadly reactive” or “broadly active” means that the H3 protein (e.g., the H3 HA protein sequence) is immunogenic and contains a diversity of epitopes (antigenic determinants; pan-epitopic) that elicit in a subject an immune response (e.g., neutralizing antibodies directed against the diversity of H3 virus HA epitopes, frequently accompanied by a T-cell response) sufficient to treat disease or infection, and/or to inhibit, neutralize, or prevent infection, caused by most or all H3 influenza viruses within a specific subtype. In embodiments, the broadly reactive, H3 virus-derived antigen protein, e.g., HA protein, is capable of eliciting a protective immune response against most or all known H3 influenza virus isolates, such as about 80%, about 85%, about 90%, about 95%, or about 96%-99% of the known H3 influenza virus isolates.

In some embodiments, the method of generating a broadly reactive, pan-epitopic protein sequence derived from H3 influenza virus includes obtaining the amino acid sequences of a group of H3 influenza virus isolates. In the case of H3 influenza, for example, the group can contain H3 influenza virus isolates from a specific subtype (such as, for example, H3N2), and/or from one or more clades/sub-clades of the specific subtype.

In embodiments, the amino acid sequences for each geographic location are aligned to generate an H3 primary sequence for each geographical region. Grouping H3 virus isolates by geographical region controls for single outbreak dominance and incomplete reporting and sequencing. A primary sequence can be generated, for example, by performing sequence analysis using AlignX (Vector NTI), or by any other method known in the art. The primary geographically-based sequences of sequence similarity and/or identity are then aligned, phylogenetic clusters of sequences are generated, followed by the generation of secondary sequences. The secondary sequences are then aligned to generate the pan-epitopic, broadly reactive, sequence in accordance with the method (see, e.g., FIG. 1). In a particular embodiment, a broadly reactive, pan-epitopic H3 influenza virus polypeptide sequence is optimized for expression in mammalian cells, for example, by reverse translation of the broadly reactive H3 influenza virus polypeptide sequence to generate a coding sequence, followed by codon-optimization and/or optimization of the RNA (such as for stability).

### Compositions and Pharmaceutical Compositions for Administration

Compositions comprising a broadly reactive, pan-epitopic H3 influenza HA protein, or a fusion protein or VLP comprising such a broadly reactive H3 influenza HA protein produced by the methods as described herein are provided. In some embodiments, the compositions further comprise a pharmaceutically acceptable carrier, excipient, or vehicle. In some embodiments, an adjuvant (a pharmacological or immunological agent that modifies or boosts an immune response, e.g. to produce more antibodies that are longer-lasting) is also employed. For example, without limitation, the adjuvant can be an inorganic compound, such as alum, aluminum hydroxide, or aluminum phosphate; mineral or paraffin oil; squalene; detergents such as Quil A; plant saponins; Freund's complete or incomplete adjuvant, a biological adjuvant (e.g., cytokines such as IL-1, IL-2, or IL-12); bacterial products such as killed Bordetella pertussis, or toxoids; or immunostimulatory oligonucleotides (such as CpG oligonucleotides).

Compositions and preparations (e.g., physiologically or pharmaceutically acceptable compositions) containing the non-naturally occurring, broadly reactive, pan-epitopic H3 influenza HA polypeptides and H3 influenza virus-like particles (VLPs) for parenteral administration include, without limitation, sterile aqueous or non-aqueous solutions, suspensions, and emulsions. Nonlimiting examples of non-aqueous solvents include propylene glycol, polyethylene glycol, vegetable oils, such as olive oil and canola oil, and injectable organic esters, such as ethyl oleate. Aqueous carriers include water, alcoholic/aqueous solutions, emulsions or suspensions, including saline and buffered media. Parenteral vehicles include, for example, sodium chloride solution, Ringer's dextrose, dextrose and sodium chloride, lactated Ringer's, or fixed oils. Intravenous vehicles include, for example, fluid and nutrient replenishers, electrolyte replenishers (such as those based on Ringer's dextrose), and the like. Preservatives and other additives may also be present in such compositions and preparations, such as, for example, antimicrobials, antioxidants, chelating agents, colorants, stabilizers, inert gases and the like.

Some of the compositions may potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids, such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

Provided herein are pharmaceutical compositions which include a therapeutically effective amount of a non-naturally occurring, broadly reactive, pan-epitopic H3 virus protein antigen, or H3 influenza VLPs alone or in combination with a pharmaceutically acceptable carrier. Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid or aqueous solution, suspension, emulsion, dispersion, tablet, pill, capsule, powder, or sustained release formulation. A liquid or aqueous composition can be lyophilized and reconstituted with a solution or buffer prior to use. The composition can be formulated as a suppository, with traditional binders and carriers such as triglycerides. Oral formulations can include standard carriers, such as pharmaceutical grades of mannitol, lactose, starch, magnesium stearate, sodium saccharine, cellulose, and magnesium carbonate. Any of the commonly known pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used in the compositions and administration methods as described are normal saline and sesame oil.

### Methods of Treatment, Administration and Delivery

Methods of treating a disease or infection, or symptoms thereof, caused by H3 influenza virus are provided. The methods comprise administering a therapeutically effective amount of a broadly reactive, pan-epitopic immunogen produced by the methods described herein or a pharmaceutical composition comprising the immunogen, or a vaccine (e.g., a VLP vaccine) as described herein to a subject (e.g., a mammal), in particular, a human subject). In an embodiment, more than one broadly reactive immunogen may be components in an immunogenic composition. One embodiment involves a method of treating a subject suffering from, or at risk of or susceptible to disease or infection, or a symptom thereof, caused by H3 influenza virus. The method includes administering to the subject (e.g., a mammalian subject), an amount or a therapeutic amount of an immunogenic composition or a vaccine comprising a non-naturally occurring, broadly reactive, pan-epitopic, H3 virus antigen polypeptide, such as HA polypeptide, or HA polypeptide VLPs, sufficient to treat the disease, infection, or symptoms thereof, caused by H3 influenza virus under conditions in which the disease, infection, and/or the symptoms thereof are treated.

In an embodiment, the methods herein include administering to the subject (including a human subject identified as in need of such treatment) an effective amount of a non-naturally occurring, broadly reactive, pan-epitopic, H3 virus antigen polypeptide, such as H3 virus HA polypeptide, or a vaccine, or a composition as described herein to produce such effect. The treatment methods are suitably administered to subjects, particularly humans, suffering from, having, susceptible to, or at risk of having a disease, disorder, infection, or symptom thereof, namely, flu or influenza. Identifying a subject in need of such treatment can be based on the judgment of the subject or of a health care professional and can be subjective (e.g. opinion) or objective (e.g. measurable by a test or diagnostic method). Briefly, the determination of those subjects who are in need of treatment or who are “at risk” or “susceptible” can be made by any objective or subjective determination by a diagnostic test (e.g., genetic test, enzyme or protein marker assay), marker analysis, family history, and the like, including an opinion of the subject or a health care provider. The non-naturally occurring, broadly reactive, pan-epitopic H3 immunogens, such as H3 HA polypeptide immunogens and vaccines as described herein, may also be used in the treatment of any other disorders in which infection or disease caused by H3 influenza virus may be implicated. A subject undergoing treatment can be a non-human mammal, such as a veterinary subject, or a human subject (also referred to as a “patient”).

In addition, prophylactic methods of preventing or protecting against a disease or infection, or symptoms thereof, caused by H3 influenza virus are provided. Such methods comprise administering a therapeutically effective amount of a pharmaceutical composition comprising an H3 vaccine (e.g., an H3 VLP vaccine) as described herein to a subject (e.g., a mammal such as a human), in particular, prior to infection of the subject or prior to onset of the disease, such as H3 virus-associated disease.

In another embodiment, a method of monitoring the progress of an H3 virus infection or disease caused by H3 virus, or monitoring treatment of the H3 infection or disease is provided. The method includes determining a level of a diagnostic marker or biomarker (e.g., an H3 virus protein, such as H3 HA), or a diagnostic measurement (e.g., screening assay or detection assay) in a subject suffering from or susceptible to infection, disease or symptoms thereof associated with H3 influenza virus, in which the subject has been administered an amount (e.g., a therapeutic amount) of a non-naturally occurring, broadly reactive, pan-epitopic H3 virus HA protein generated according to the methods described herein, or a vaccine as described herein sufficient to treat the infection, disease, or symptoms thereof. The level or amount of the marker or biomarker (e.g., protein) determined in the method can be compared to known levels of the marker or biomarker in samples from healthy, normal controls; in a pre-infection or pre-disease sample of the subject; or in other afflicted/infected/diseased patients to establish the treated subject's disease status. For monitoring, a second level or amount of the marker or biomarker in in a sample obtained from the subject is determined at a time point later than the determination of the first level or amount, and the two marker or biomarker levels or amounts can be compared to monitor the course of disease or infection, or the efficacy of the therapy/treatment. In certain embodiments, a pre-treatment level of the marker or biomarker in the subject (e.g., in a sample obtained from the subject) is determined prior to beginning treatment as described; this pre-treatment level of marker or biomarker can then be compared to the level of the marker or biomarker in the subject after the treatment commences and/or during the course of treatment to determine the efficacy of (monitor the efficacy of) the disease treatment.

The non-naturally occurring, broadly reactive, pan-epitopic, H3 virus antigen polypeptides, such as H3 virus HA polypeptides generated in accordance with the described methods, and VLPs comprising H3 HA polypeptides, or compositions thereof, can be administered to a subject by any of the routes normally used for introducing a recombinant protein, composition containing the recombinant protein, or recombinant virus into a subject. Routes and methods of administration include, without limitation, intradermal, intramuscular, intraperitoneal, intrathecal, parenteral, such as intravenous (IV) or subcutaneous (SC), vaginal, rectal, intranasal, inhalation, intraocular, intracranial, or oral. Parenteral administration, such as subcutaneous, intravenous or intramuscular administration, is generally achieved by injection. Injectables can be prepared in conventional forms and formulations, either as liquid solutions or suspensions, solid forms (e.g., lyophilized forms) suitable for solution or suspension in liquid prior to injection, or as emulsions. Injection solutions and suspensions can be prepared from sterile powders, granules, and tablets. Administration can be systemic or local.

The non-naturally occurring, broadly reactive, pan-epitopic, H3 virus polypeptides, such as H3 virus HA polypeptides generated in accordance with the described methods, and VLPs comprising H3 HA polypeptides, or compositions thereof, can be administered in any suitable manner, such as with pharmaceutically acceptable carriers as described supra. Pharmaceutically acceptable carriers are determined in part by the particular immunogen or composition being administered, as well as by the particular method used to administer the composition. Accordingly, a pharmaceutical composition comprising the non-naturally occurring, broadly reactive, pan-epitopic, H3 virus antigen polypeptides, such as H3 virus HA polypeptides, and VLPs comprising H3 HA polypeptides, or compositions thereof, can be prepared using a wide variety of suitable and physiologically and pharmaceutically acceptable formulations.

Administration of the broadly reactive, pan-epitopic, H3 virus antigen polypeptides, such as H3 virus HA polypeptides, generated in accordance with the described methods, and VLPs comprising HA polypeptides, or compositions thereof, can be accomplished by single or multiple doses. The dose administered to a subject should be sufficient to induce a beneficial therapeutic response in a subject over time, such as to inhibit, reduce, ameliorate, or prevent disease or infection by H3 influenza virus. The dose required will vary from subject to subject depending on the species, age, weight and general condition of the subject, by the severity of the infection being treated, by the particular composition being used and by the mode of administration. An appropriate dose can be determined by a person skilled in the art, such as a clinician or medical practitioner, using only routine experimentation.

Further provided is a method of eliciting an immune response to H3 influenza virus in a subject by administering to the subject a non-naturally occurring, broadly reactive, pan-epitopic, H3 influenza HA protein disclosed herein, fusion proteins containing the H3 influenza HA protein, VLPs containing the influenza HA protein, or compositions thereof as described herein. In some embodiments, the H3 HA protein, HA fusion protein or VLP can be administered using any suitable route of administration, such as, for example, by intramuscular injection. In some embodiments, the H3 HA protein, fusion protein, or VLP is administered as a composition comprising a pharmaceutically acceptable carrier. In some embodiments the composition comprises an adjuvant selected from, for example, alum, Freund's complete or incomplete adjuvant, a biological adjuvant or immunostimulatory oligonucleotides (such as CpG oligonucleotides). In other embodiments, the composition may be administered in combination with another therapeutic agent or molecule.

Also provided is a method of immunizing a subject against infection or disease or the symptoms thereof caused by the H3 influenza virus, in which the method involves administering to the subject VLPs containing a non-naturally occurring, pan-epitopic, broadly reactive H3 influenza HA protein generated according to the described methods or administering a composition thereof. In some embodiments of the method, the composition further comprises a pharmaceutically acceptable carrier and/or an adjuvant. For example, the adjuvant can be alum, Freund's complete or incomplete adjuvant, a biological adjuvant or immunostimulatory oligonucleotides (such as CpG oligonucleotides). In an embodiment, the H3 VLPs (or compositions thereof) are administered intramuscularly.

In some embodiments of the methods of eliciting an immune response or immunizing a subject against virus infection or disease caused by or associated with H3 influenza virus, the subject is administered at least 1 μg of the VLPs containing a non-naturally occurring, broadly reactive, pan-epitopic H3 virus HA protein, such as at least 5 at least 10 at least 15 at least 20 at least 25 at least 30 at least 40 μs g or at least 50 μg of the VLPs containing the non-naturally occurring, broadly reactive, pan-epitopic H3 virus HA protein, for example about 1 to about 50 μg or about 1 to about 25 μg of the VLPs containing the H3 HA protein. In particular examples, the subject is administered about 5 to about 20 μg of the VLPs, or about 10 to about 15 μg of the VLPs. In a specific, yet nonlimiting example, the subject is administered about 15 μg of the VLPs. However, one of skill in the art is capable of determining a therapeutically effective amount of VLPs (for example, an amount that provides a therapeutic effect or protection against H3 influenza virus infection) suitable for administering to a subject in need of treatment or protection from virus infection.

It is expected that the administration of immunogens, immunogenic compositions and VLPs comprising a non-naturally occurring, broadly reactive, pan-epitopic H3 HA protein (or encoding polynucleotide) generated by the methods described herein will elicit high titers of neutralizing antibodies directed against the diverse repertoire of epitopic determinants on the H3 HA protein immunogen, as well as protective levels of H3 HA-inhibiting (HAI) antibodies that are directed against a number of representative H3 isolates and will provide complete protection against lethal challenge with H3 virus and related types of H3 virus. The immunogens, immunogenic compositions and VLPs containing a non-naturally occurring, broadly reactive, pan-epitopic H3 influenza HA protein generated by the methods as described herein elicit a broader immune response (e.g., elicit neutralizing antibodies directed against a broader range of H3 virus isolates) compared to the immune response elicited by a polyvalent H3 influenza virus vaccine, which typically contains a preparation of two or more strains, types, subtypes, or isolates of the H3 virus.

Accordingly, the non-naturally occurring, broadly reactive, pan-epitopic immunogens provided by the methods described herein provide a beneficial advantage over polyvalent vaccine preparations, both in terms of treatment and cost effectiveness. By their pan-epitopic nature, the broadly reactive H3 virus antigens and antigen sequences generated by the methods described herein provide a specific, unique immunogen, immunogenic composition, or vaccine for eliciting an immune response against different H3 virus types and subtypes (and related viruses) in a recipient subject. This stands in contrast to polyvalent or multivalent vaccines which are typically prepared from two or more (multiple) strains, isolates, or subtypes of viruses, e.g., H3, which do not provide the diversity and universality of antigenic determinants (epitopes) as provided by the specifically-generated immunogens described herein, and which are more labor intensive and costly to produce compared with the presently described methods.

### Adjuvants and Combination Therapies

The H3 virus immunogens or immunogenic compositions containing an H3 protein antigen (e.g., an H3 HA antigen) generated by the described methods, or containing H3 VLPs as described herein, can be administered alone or in combination with other therapeutic agents to enhance antigenicity or immunogenicity, i.e., to increase an immune response, such as the elicitation of specific antibodies, in a subject. For example, the H3 influenza VLPs can be administered with an adjuvant, such as alum, Freund's incomplete adjuvant, Freund's complete adjuvant, biological adjuvant, or immunostimulatory oligonucleotides (such as CpG oligonucleotides).

One or more cytokines, such as interleukin-1 (IL-2), interleukin-6 (IL-6), interleukin-12 (IL-12), the protein memory T-cell attractant “Regulated on Activation, Normal T Expressed and Secreted” (RANTES), granulocyte-macrophage-colony stimulating factor (GM-CSF), tumor necrosis factor-alpha (TNF-α), or interferon-gamma (IFN-γ); one or more growth factors, such as GM-CSF or granulocyte-colony stimulation factor (G-CSF); one or more molecules such as the TNF ligand superfamily member 4 ligand (OX40L) or the type 2 transmembrane glycoprotein receptor belonging to the TNF superfamily (4-1BBL), or combinations of these molecules, can be used as biological adjuvants, if desired or warranted (see, e.g., Salgaller et al., 1998, J. Surg. Oncol. 68(2):122-38; Lotze et al., 2000, Cancer J. Sci. Am. 6(Suppl 1):S61-6; Cao et al., 1998, Stem Cells 16(Suppl 1):251-60; Kuiper et al., 2000, Adv. Exp. Med. Biol. 465:381-90). These molecules can be administered systemically (or locally) to a subject.

Several ways of inducing cellular responses, both in vitro and in vivo, are known and practiced in the art. Lipids have been identified as agents capable of assisting in priming cytotoxic lymphocytes (CTL) in vivo against various antigens. For example, palmitic acid residues can be attached to the alpha and epsilon amino groups of a lysine residue and then linked (for example, via one or more linking residues, such as glycine, glycine-glycine, serine, serine-serine, or the like) to an immunogenic peptide (U.S. Pat. No. 5,662,907). The lipidated peptide can then be injected directly in a micellar form, incorporated in a liposome, or emulsified in an adjuvant. As another example, E. coli lipoproteins, such as tripalmitoyl-S-glycerylcysteinlyseryl-serine can be used to prime tumor-specific CTL when covalently attached to an appropriate peptide (see, e.g., Deres et al., 1989, Nature 342:561). Moreover, the induction of neutralizing antibodies can also be primed with the same molecule conjugated to a peptide which displays an appropriate epitope, and two compositions can be combined to elicit both humoral and cell-mediated responses where such a combination is deemed desirable.

While treatment methods may involve the administration of VLPs containing a non-naturally occurring, broadly reactive, pan-epitopic H3 HA immunogenic protein generated by the methods described herein, one skilled in the art will appreciate that the non-naturally occurring, broadly reactive, pan-epitopic H3 influenza HA protein itself (in the absence of a viral particle), as a component of a pharmaceutically acceptable composition, or as a fusion protein, can be administered to a subject in need thereof to elicit an immune response in the subject.

### Kits

Also provided are kits containing a non-naturally occurring, broadly reactive, pan-epitopic H3 virus immunogen generated by the described methods, or a vaccine, or a pharmaceutically acceptable composition containing the immunogen and a pharmaceutically acceptable carrier, diluent, or excipient, for administering to a subject, for example. Kits containing one or more of the plasmids, or a collection of plasmids as described herein, are also provided. As will be appreciated by the skilled practitioner in the art, such a kit may contain one or more containers that house the immunogen, vaccine, or composition, diluents or excipients, as necessary, and instructions for use.

### Recombinant Polypeptide Expression

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### EXAMPLES

The following examples are provided to illustrate certain particular features and/or embodiments. The examples should not be construed to limit the disclosure to the particular features or embodiments described.

### Example 1

### Method for Generating a Seasonal, Broadly Reactive, Pan-Epitopic H3 HA Antigen Based on Drift Variants

The steps of the method of generating a non-naturally occurring, broadly reactive, immunogenic H3 influenza HA antigen based on human flu seasons as described herein are summarized below. A general depiction of the method is shown in FIG. 1. Optimally, the results of some or all of the steps of the method may be displayed in a visual form on a display device.

A. Obtain H3 antigen (e.g., HA) sequences from an online database (flu database), e.g., via the Global Initiative on Sharing All Influenza Data (“GISAID”) or the Influenza Research Database (“FluDB”), saving as FASTA amino acid sequences with accession ID information, and analyze HA (or NA) amino acid sequences from numerous H3 virus strains (e.g., 6,430 HA amino acid sequences) over a given time period (e.g., 1968-2013). For example, such a search query can include “Northern Hemisphere: 10/1/______ (month/date/year)-4/30/______+1 (month/date/year+1);” “Southern Hemisphere: 5/1/______-9/31/______;” “Yearly: 1/1/______-12/31/______.”

B. Assemble the multiple sequences and perform alignments for the sequences over the demarcated time period and geographical location using MUSCLE (MUltiple Sequence Comparison by LogExpectation) alignment (Clustering NJ, Sequence weighting scheme CLUSTALW), which is faster than Clustal and provides accurate alignments.

C. Eliminate incomplete H3 HA sequences (retain HA1 (HA head region sequences), amino acids 20-300 of the complete HA amino acid sequence) and remove partial sequences, for example, sequences of less than 280 residues, or sequences with gaps or ambiguities.

D. Assemble a phylogenetic tree by performing sequence alignments, based on the parameters: Jukes-Cantor genetic distance model, Neighbor joining phylogenetic tree building method, and no outgroup. For the phylogenetic tree, select the farthest upstream node to each defined clade or branch. While “node” and “cluster” are selected, color the node with a unique color. The parameters, phylogenetic trees and/or sequences therein are optimally displayed in a visual form on a display device.

E. Transform branches to “cladogram,” max characters “30,” and “Enable all layouts for unrooted trees.” The cladogram can be displayed in a visual form on a display device.

F. Select branches comprising multiple aligned sequences from those phylogenetic trees that have >98% sequence identity and demarcate the tree branches with color/cluster ID. Note whether substitution per site rate distance is >0.001. Clustering parameters are revised to have more stringent criteria, i.e., sequence similarity within the antigenic regions or specific antigenic sites.

G. Extract cluster(s) with substitution per site of >0.001 and use the cluster(s) to generate a primary H3 sequence of sequence similarity and/or identity for building future secondary sequences having sequence similarity and/or identity. Repeat steps F and G until primary H3 sequences have been generated for an entire tree from step E.

H. Generate an alignment and phylogenetic tree of all H3 primary sequences from step G (repeat as necessary). The radial phylogenetic tree generated to identify clusters of H3 antigen (e.g., HA) sequences or clusters of H3 HA sequences in a given geographic location (e.g., Southern Hemisphere) in a season (e.g., 2002), and/or selected branches of sequences from the phylogenetic tree can be displayed in a visual form on a display device.

I. Designate clusters of +5 primary sequences and extract sequences of similarly and/or identity (redefine/color) as a secondary cluster;

J. Generate a phylogenetic tree of H3 secondary sequences of similarity and/or identity from step I and assess whether multiple branches with substitution per site of >0.001 to assess variability among the sequences;

K. Combine multiple branches if the substitution rate per site distance is <0.001 and generate a sequence having sequences of similarity and/or identity from the cluster to produce an H3 tertiary sequence;

L. Repeat for each time period of primary sequences, i.e., Scenario 1: 2009-2012, 2009-2013 (or 2010-2013), 2009-2014 (or 2011-2014), or Scenario 2: 2009-2012, 2010-2013 (or 2009-2013), 2011-2014 (or 2009-2014).

FIGS. 2A-2C depict aspects of this method.

The method further includes translating the output of one or more of the various steps of the method into a visual form such as displaying the output on a display device.

### Example 2

### Construction and Synthesis of Pan-Epitopic H3 HA Antigens for Use as Immunogens to Elicit Broadly Reactive Antibodies

This Example describes the generation of broadly reactive, pan-epitopic H3 HA antigen sequences using the method described herein. In general, a backbone sequence representing H3 HA amino acid sequences from the time period 1996-1999 was generated by the method as described herein to arrive at a broadly reactive, pan-epitopic sequence that captures HA epitopes from past and present seasons.

Influenza A HA protein sequences from 1,401 human H3N2 infections collected from May 1, 1996-Sep. 30, 1999 were downloaded from the Global Initiative on Sharing Avian Influenza Data (GISAID) database, organized by date of isolation and categorized by each Northern (October 1 to April 30) or Southern Hemisphere (May 1 to September 30) influenza season. HA sequences were clustered for each of the 3 Southern Hemisphere influenza seasons, and for the two Northern Hemisphere seasons within this time frame. For each round of sequence similarity/identity generation, multiple sequence alignment was performed using the GENEIOUS® MUSCLE alignment method, and Jukes-Cantor phylogenetic neighbor-joining, no out-grouping, unrooted circular trees (sequence clusters) were constructed. HA sequences representing amino acids 20-300 of the HA1 head region were aligned for each cluster, and the most common residue found among a designated set of virus HA sequences was used to yield the primary sequence. Ambiguities were avoided through alignment of ≥3 sequences. Partial sequences were excluded from the analysis.

Multiple rounds of primary sequence assembly from within a season were layered together to yield secondary sequences for that specific influenza season. This methodology was used for each of the 5 influenza seasons during the time period (May 1, 1996-Sep. 30, 1999), which yielded 3 secondary sequences. These secondary HA1 sequences were aligned in a GENEIOUS® MUSCLE alignment method, and Jukes-Cantor phylogenetic neighbor-joining, no out-grouping, unrooted linear trees were constructed. A tertiary (3°) HA1 sequence was determined and was termed the backbone sequence comprising the 1996 to 1999 time period. A flowchart of the method is shown in FIG. 1.

Additional next generation HA1 head region sequences (i.e., HA1 sequences expected to reflect a composite of epitopes (antigenic determinants)) from viral antigen sequences representing viruses from different seasons and from different geographic locations) were developed by one of three scenarios (Scenario #1, #2, or #3). 313 HA sequences isolated during the 1996 Southern Hemisphere influenza season (May 1, 1996-Sep. 30, 1999) were downloaded. The same methodology that resulted in an unrooted circular tree alignment with 8 primary clusters, which, when layered together, produced 2 new, secondary HA sequences from this season.

In Scenario #1, these 2 new, secondary sequences were added to the previously generated secondary backbone linear phylogenetic alignment, and the 4 oldest secondary sequences that were produced from the 1996 Southern Hemisphere isolates (May 1, 1996-Sep. 30, 1996) were eliminated from the alignment. All 12 secondary HA sequences (Oct. 1, 1996-Sep. 30, 1997) were weighted equally and resulted in a single tertiary HA sequence. (FIGS. 2A-2C).

For Scenario #2, the same 2 new, secondary sequences were added to the previously generated secondary backbone linear phylogenetic alignment, but unlike Scenario #1, none of the older secondary sequences (May 1, 1996-Sep. 30, 1996) were eliminated from the alignment; instead, the older secondary sequences extended the era included to capture an additional season, and increased the number of secondary sequences used to generate the final tertiary HA sequence to 21. (FIGS. 2A and 2D).

Both Scenarios were used to include sequences from influenza season through the 1999 Southern hemisphere season. Scenario #1 produced 3 HA sequences, 1 of which was unique, and Scenario #2 produced 4 HA sequences; 1 of these tertiary HA sequences was unique. In the end, the process resulted in the generation of a total of 2 new tertiary HA sequences that were produced and designated Ross-1 and Ross-2. The same methodology was used to generate a backbone sequence using sequences from the 2002 to 2005 (May 1, 2002-Sep. 30, 2005), influenza seasons and was rolled through using both Scenarios #1 and #2 from the 2002 to 2005 Northern hemisphere seasons. For the backbone sequence using sequences from 2009 to 2011 (May 1, 2009-Sep. 30, 2011), and influenza seasons and was rolled through using both Scenarios #1 and #2 from the 2009 to 2011 Northern hemisphere seasons. And for the backbone sequences from 2011 to 2014 (May 1, 2011-Sep. 30, 2014) influenza seasons and was rolled through using both Scenarios #1 and #2 from the 2011 to 2014 Northern hemisphere seasons.

This process produced 5 unique sequences (called Ross-2). These tertiary sequences were aligned with the wild-type vaccine strains, as well as HA sequences from co-circulating variants, in a final phylogenetic tree. The 9 H3N2 HA constructs were synthesized and inserted into the pTR600 expression vector, as previously described and referenced supra. Examples of H3 HA sequences, called Ross-1 and Ross-2 (566 amino acids), generated using described method are presented in FIG. 3.

A similar approach was then used to generate a protein sequence for the HA2 tail or stalk portion (AA 301-566) of the influenza virus during the same time frame as that used to design the HA1 protein sequence. Sequences representing Northern and Southern Hemispheres were downloaded; primary sequences were generated, and phylogenetic trees were generated. Primary sequences were grouped together to form secondary sequences, and those secondary sequences were used to generate a final tertiary sequence spanning 2.5 years of influenza circulation.

For the sequences that were designed for the 1996-1999 and 2002-2005 time frames, the leader sequence (AA 1-20) from A/Wisconsin/67/2005 influenza virus was used in combination with the final broadly reactive antigen HA1 and HA2 antigen protein sequences to generate a full length (566AA sequence). For the sequences that were designed for the 2009-2015 time frame, the leader sequence (AA 1-20) from A/Texas/50/2012 influenza virus was used in combination with the final broadly reactive HA1 and HA2 antigen protein sequences to generate a full length (566 AA sequence).

### Example 3

### Hemagglutination-Inhibition (HAI) Assay

The hemagglutination inhibition (HAI) assay was used to assess functional antibodies to the HA protein that are able to inhibit agglutination of guinea pig, horse, or turkey erythrocytes (red blood cells (RBCs)). The protocols were adapted from the WHO laboratory influenza surveillance manual (Gillim-Ross and Subbarao, 2006, Clin Microbiol Rev 19(4):614-636) and use the host-species that is frequently used to characterize contemporary H3N2 strains that have preferential binding to alpha (2, 6) linked sialic acid receptors. Turkey or guinea pig erythrocytes were used to compare whether there was a difference in HAI depending on the type of erythrocyte that was used.

To inactivate nonspecific inhibitors, sera were treated with receptor-destroying enzyme (RDE) (Denka Seiken, Co., Japan) prior to being tested. (Bright et al., 2005, Lancet 366(9492):1175-1181; Bright et al., 2003, Virology 308(2):270-278; Bright et al., 2006, JAMA 295(8):891-894; Mitchell et al., 2004, Vaccine 21(9-10):902-914; Ross et al., 2000, Nat Immunol 1(2):127-131). Briefly, three parts of RDE was added to one part of sera and incubated overnight at 37° C. RDE was inactivated by incubation at 56° C. for approximately 30 minutes (˜30 min.). RDE-treated sera were diluted in a series of two-fold serial dilutions in v-bottom microtiter plates. An equal volume of each virus, e.g., H3N2 virus, adjusted to approximately 8 hemagglutination units (HAU)/50 μl, was added to each well. The plates were covered and incubated at room temperature for 20 minutes, followed by the addition of 0.8% guinea pig erythrocytes (Lampire Biologicals, Pipersville, Pa., USA) in phosphate buffered saline (PBS). Red blood cells were stored at 4° C. and used within 72 hours of preparation.

The plates were mixed by agitation and covered, and the RBCs were allowed to settle for 1 hour at room temperature. The HAI titer was determined by the reciprocal dilution of the last well that contained non-agglutinated RBCs. Positive and negative serum controls were included for each plate. All mice were negative (HAI≤1:10) for preexisting antibodies to currently circulating human influenza viruses prior to vaccination. Seroprotection was defined as HAI titer >1:40, and seroconversion was defined as a 4-fold increase in titer compared to baseline, as per the WHO and European Committee for Medicinal Products to evaluate influenza vaccines more stringent threshold of >1:80 was often examined. Because mice are naïve and seronegative at the time of vaccination, seroconversion and seroprotection rates are interchangeable in the experiments.

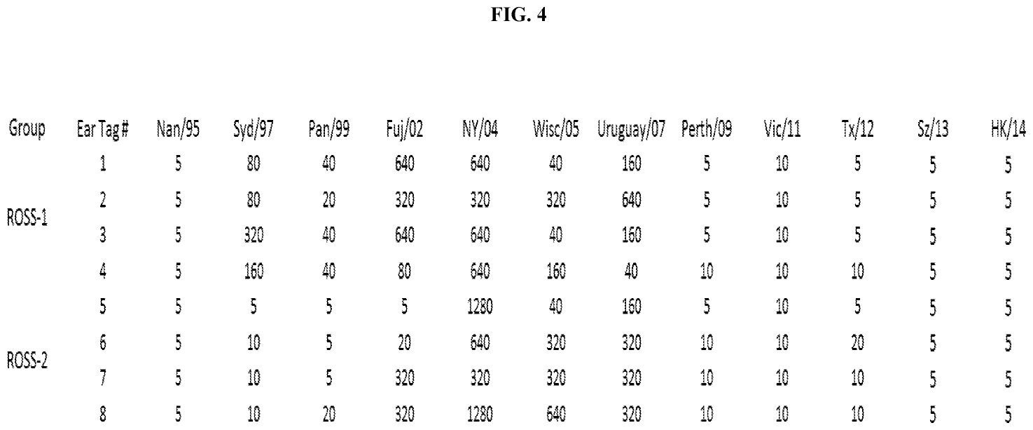

FIG. 4 shows hemagglutination inhibition antibody titers produced against different strains of H3 viruses in different seasons resulting from hemagglutination inhibition assays using the Ross-1 and Ross-2 polypeptides as immunogens administered to test animals. The antibody titers of neutralizing antibodies in the plasma samples obtained from the test animals are presented in the table.

### Example 4

### Virus-Like Particle (Vaccine) Preparation

Mammalian 293T cells were transfected with each of three mammalian expression plasmids expressing either the influenza neuraminidase (A/mallard/Alberta/24/01, H7N3), the HIV p55 Gag sequences, or one of the broadly reactive, ‘Next Generation’ HA expression plasmids (e.g., containing a polynucleotide sequence encoding an HA protein, for example, a sequence of Ross-1 or Ross-2 HA proteins as shown in FIG. 3, using previously described methods (see, e.g., US Patent Application Publication US 2015/0030628). Following 72 hours of incubation at 37° C., supernatants from transiently transfected cells were collected, centrifuged to remove cellular debris, and filtered through a 0.22 μm pore membrane. Mammalian virus-like particles (VLPs) were purified and sedimented by ultracentrifugation on a 20% glycerol cushion at 135,000×g for 4 hours at 4° C. VLPs were resuspended in phosphate buffered saline (PBS) and total protein concentration was assessed using a conventional bicinchoninic acid assay (BCA). The hemagglutination activity of each preparation of VLPs was determined by adding an equal volume of turkey red blood cells (RBCs) to a V-bottom 96-well plate and incubating with serially diluted volumes of VLPs for a 30 minute incubation at room temperature (RT). The highest dilution of VLP with full agglutination of RBCs was considered the endpoint HA titer.

### Example 5

### Determination of HA Content by Enzyme Linked Immunosorbent Assay (ELISA)

A high-affinity, 96-well, flat-bottom ELISA plate was coated with 5-10 μg of total protein of VLP and serial dilutions of a recombinant H3 antigen (3006_H3_Vc, Protein Sciences, Meriden, Conn.) in ELISA carbonate buffer (50 mM carbonate buffer, pH 9.5) were added to the wells. The plate was incubated overnight at 4° C. on a rocker. The next morning, the plates were washed in PBS with 0.05% Tween-20 (PBST), and non-specific epitopes were blocked with 1% bovine serum albumin (BSA) in PBST solution for 1 hour at RT. The buffer was removed, and stalk-specific Group 2 antibody CR8020 (Tharakaraman, K. et al., 2014, Cell Host & Microbe, Vol. 15, pp. 644-651; Ekiert, D. C. et al., 2012, Science, 333(6044):843-850; Creative Biolabs, Shirley, N.Y.) was added to plate, followed by a 1 hour incubation at 37° C. The plates were washed and then were probed with goat anti-human IgG horseradish-peroxidase-conjugated secondary antibody (2040-05, Southern Biotech, Birmingham, Ala.) for 1 hour at 37° C.

The plates were washed. Freshly prepared o-phenylenediamine dihydrochloride (OPD) (P8287, Sigma, City, State, USA) substrate in citrate buffer (P4922, Sigma) was then added to wells, followed by the addition of 1N H2504 stopping reagent. The plates were read at 492 nm absorbance using a microplate reader (Powerwave XS, Biotek, Winooski, Vt.). Background signal was subtracted from negative wells. Linear regression standard curve analysis was performed using the known concentrations of recombinant standard antigen to estimate the HA content in lots of VLPs.

### Example 6

### Mouse and Ferret Studies

### Mouse Studies

BALB/c mice (Mus musculus, females, 6 to 8 weeks old) were purchased from Jackson Laboratory (Bar Harbor, Me., USA), housed in microisolator units and allowed free access to food and water. The animals were cared for under University of Georgia Research Animal Resources guidelines for laboratory animals. All procedures were reviewed and approved by the Institutional Animal Care and Use Committee (IACUC). Mice (5 or 10 mice per group) were vaccinated with purified virus-like particles (VLPs), (3.0 μg/mouse), based upon HA content from the ELISA quantification, and VLP vaccines were delivered to the animals via intramuscular injection at week 0. Animals were boosted with the same vaccine at the same dose at weeks 4 and 8. Vaccines at each dose were formulated with an emulsified squalene-in-water adjuvant (Sanofi Pasteur, Lyon, France). The final concentration after mixing 1:1 with VLPs is 2.5% squalene. Twenty-eight days after each vaccination, blood samples were collected via the submandibular cheek, and the samples were transferred to a microcentrifuge tube. The tubes were centrifuged at 10,000 rpm for 10 minutes. Serum samples were removed and frozen at −20° C.±5° C.

### Ferret Studies

Fitch ferrets (Mustela putorius faro, female, 6-12-months of age), influenza naive and de-scented, were purchased from Marshall Farms (Sayre, Pa., USA). Ferrets were pair-housed in stainless steel cages (Shor-line, Kansas City, Kans., USA) containing Sani-chips Laboratory Animal Bedding (P.J. Murphy Forest Products, Montville, N.J., USA). Ferrets were provided with Teklad Global Ferret Diet (Harlan Teklad, Madison, Wis., USA) and fresh water ad libitum.

The purified VLPs were diluted in PBS, pH 7.2, to achieve final concentration. Ferrets (n=3) were vaccinated with 15 μg of purified VLPs, based upon HA content as determined by densitometry assay, via intramuscular injection in the quadriceps muscle in a volume of 0.25 ml at week 0, and then were boosted with the same dose at week 3. Vaccines were stored at −80° C. prior to use and formulated with IMJECT® alum adjuvant (IMJECT® Alum; Pierce Biotechnology, Rockford, Ill. USA) or with the above-described emulsified squalene-in-water adjuvant immediately prior to use. Animals were monitored for adverse events including weight loss, temperature, loss of activity, nasal discharge, sneezing and diarrhea weekly during the vaccination regimen. Prior to vaccination, animals were confirmed by HAI assay to be seronegative for circulating influenza A (e.g., H1N1) and influenza B viruses. Fourteen to twenty-one days after each vaccination, blood was collected from anesthetized ferrets via the anterior vena cava and transferred to a microfuge tube. The tubes were centrifuged; serum was removed and frozen at −20±5° C.

### Example 7

### Next-Generation Methodology to Improve Upon and Advance the Generation of Existing Pathogen-Derived Antigen Sequences and Produce Seasonal, Broadly Reactive, Pan-Epitopic H3 HA Antigen Based on Drift Variants

The next-generation methodology described in this Example was developed to improve upon and advance the generation of existing pathogen-derived antigen sequences, (e.g., influenza virus HA), produced by prior computationally optimized methods. The next-generation methodology involved the generation of pathogen-derived antigen sequences to produce immunogenic sequences in a real-time fashion at selected time periods, e.g., every six months; so as to include new antigen sequences as new pathogenic strains emerged in the wild; and to incorporate future antigen sequences of emerging pathogens (e.g., influenza viruses) into a final pan-epitopic antigen sequence for use as an immunogen. The method described below represents a next-generation technology for generating broadly reactive antigen sequences from the HA1 portion of influenza virus for use as immunogens.

In the method, a backbone sequence was created by downloading sequences from public databases, e.g., GISAID or FluDB, within a given time period, e.g., a six-month period, for a given subtype, e.g., H3N2. Following alignment of the sequences, the HA1 portion (amino acids 20-300) was typically extracted, and a phylogenetic tree was created. The tree was divided into different primary clusters. Each cluster contained sequences that were at least about 98% identical to each other, and a primary sequence was generated from each cluster. This process was repeated until the entire phylogenetic tree was divided into multiple primary sequences, which were then realigned to one another. A new phylogenetic tree was created using only the primary sequences. Three or more primary sequences that were located in close proximity to each other in the tree were then extracted, aligned, and a new secondary sequence was formed. These steps were repeated until all of the primary sequences were utilized to generate multiple secondary sequences.

The above-described steps were repeated for the following six-month period until a series of five consecutive, 6-month periods were captured. All of the secondary sequences from a given 2.5-year period were then aligned; a phylogenetic tree was created, and the sequences that were close to each other in identity were used to generate multiple tertiary sequences. The tertiary sequences were aligned, and all of the tertiary sequences were used to generate a final quaternary backbone sequence.

Once a backbone sequence was generated for the antigen, new, emerging sequences were added in an effort to “roll” the design forward in time. By way of example, two different procedures were used to roll forward a new antigen design. In Scenario 1 (FIG. 2A), secondary sequences were added in from the most recent six-month period, and secondary sequences were ‘dropped off’ from the oldest six-month period used to construct the backbone sequence. After the first six months of rolling, the resulting antigen sequence was comprised of multiple secondary sequences that encompassed the most recent 2.5-year period. In Scenario 2 (and 3) (FIG. 2A), secondary sequences were added in from the most recent six-month period, and all of the previous secondary sequences from the generated backbone sequence were retained. After the first six months of rolling, the resulting antigen sequence was comprised of multiple secondary sequences that covered the most recent three-year period.

Similarly, an antigenic immunogen sequence for the HA2 portion of hemagglutinin (HA2), (amino acids 301-566) during the same time frame was generated using the above approach. Sequences were obtained from a six-month time frame; primary sequences were generated using the at least about 98% sequence identity criterion described above; similar primary sequences were grouped together to produce secondary sequences; and the secondary sequences were used to generate tertiary sequences having sequence similarity and/or identity. Lastly, all of the tertiary sequences that spanned 2.5 years of HA sequence coverage were aligned and a quaternary backbone HA antigenic immunogen was generated. The quaternary HA1 and HA2 sequences were combined and coupled to a leader sequence (typically NH-terminal amino acids 1-19) that was derived from a wild-type strain of influenza virus to form the amino acid sequence of the pan-epitopic, broadly reactive antigenic immunogen.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of generating a non-naturally occurring, pan-epitopic immunogen for generating an immune response against present and future H3 Influenza virus strains in a subject, the method comprising:
(a) generating a phylogenetic tree comprising full length related antigen sequences derived from H3 strains present during two or more consecutive flu seasons in the Northern and Southern Hemispheres;
(b) identifying clusters of antigen sequences within the tree, each cluster having at least 95% identity and at least about 0.001 substitution per site relative to the other sequences within the cluster;
(c) generating for each cluster a non-naturally occurring primary sequence comprising amino acids that are conserved or identical within the cluster;
(d) generating a phylogenetic tree comprising the primary sequences of step (c);
(e) selecting three or more clusters of antigen sequences within the primary sequences, each selected cluster comprising at least about 0.001 amino acid substitutions per site and generating secondary sequences comprising amino acids that are conserved or identical within the three or more clusters;
(f) generating a phylogenetic tree comprising the secondary sequences, wherein branches of the tree are combined if the substitution rate per amino acid site distance is less than about 0.001 to produce a plurality of tertiary sequences;
(g) generating a quaternary backbone sequence comprising amino acids that are conserved or identical among the tertiary sequences; and
(h) generating a non-naturally occurring, pan-epitopic immunogen by incorporating secondary sequences from step (e), wherein the selected sequences are derived from the most recent time period.

2. The method of claim 1, wherein, in step (h), the most recent time period is a 6-month time period.

3. The method of claim 1, wherein step (d) and step (e) are optional.

4. The method of claim 1, wherein steps (a)-(c) are repeated two or more times.

5. The method of claim 1, wherein steps (b) and (c) are repeated two or more times prior to step (d).

6. The method of claim 1, wherein steps (e) and (f) are repeated two or more times.

7. A method of generating a non-naturally occurring, pan-epitopic immunogen for generating an immune response against present and future H3 Influenza virus strains in a subject, the method comprising:
(a) generating a phylogenetic tree comprising full length related antigen sequences derived from H3 strains present during two or more consecutive recent flu seasons in the Northern and Southern Hemispheres within a six-month time period;
(b) identifying clusters of antigen sequences within the tree, each cluster having at least 95% identity and at least about 0.001 substitution per site relative to the other sequences within the cluster;
(c) generating for each cluster a non-naturally occurring primary sequence comprising amino acids that are conserved or identical within the cluster;
(d) generating a phylogenetic tree comprising the primary sequences of step (c);
(e) selecting three or more clusters of antigen sequences within the primary sequences, each selected cluster comprising at least about 0.001 amino acid substitutions per site and generating secondary sequences comprising amino acids that are conserved or identical within the three or more clusters;
(f) repeating steps (a)-(e) until secondary sequences from a series of recent consecutive six-month time periods have been selected over a preselected total time period;
(g) generating a phylogenetic tree comprising the secondary sequences, wherein branches of the tree are combined if the substitution rate per amino acid site distance is less than about 0.001 to produce a plurality of tertiary sequences;
(h) generating a quaternary backbone sequence comprising amino acids that are conserved or identical among the tertiary sequences; and
(i) generating a non-naturally occurring, pan-epitopic immunogen by incorporating secondary sequences from step (e) into the quaternary backbone sequence, wherein
(i) secondary sequences from the most recent six-month time period are incorporated into the backbone sequence and secondary sequences from the oldest six-month time period are eliminated from the backbone sequence, thereby producing a sequence comprising multiple secondary sequences spanning the preselected total time period; or
(ii) secondary sequences from the most recent six-month time period and from the oldest six-month time period are incorporated into the backbone sequence, thereby producing a sequence comprising multiple secondary sequences spanning the preselected total time period.

8-21. (canceled)

22. The method of claim 1, further comprising:
generating at least 2 H3 secondary antigen sequences for a disease season of time based on the phylogenetic tree sequences generated in step (f);
adding the at least 2 H3 secondary sequences to a backbone sequence based on clustered antigen sequences in the season;
eliminating from the backbone sequence older secondary antigen sequences produced from a predetermined disease season in a geographic location; and
generating a pan-epitopic H3 immunogen by equally weighting multiple secondary antigen sequences.

23. The method of claim 1, further comprising:
generating at least 2 H3 secondary antigen sequences for a disease season of time based on the phylogenetic tree sequences generated in step (f);
adding the at least 2 H3 secondary sequences to a backbone sequence based on clustered antigen sequences in the season;
retaining in the backbone sequence antigen sequences produced from a predetermined disease season in a geographic location to include an additional season; and
generating a pan-epitopic H3 immunogen by equally weighting multiple secondary antigen sequences.

24-28. (canceled)

29. A non-naturally occurring immunogen generated using the method of claim 1.

30. An immunogenic composition or vaccine comprising the non-naturally occurring immunogen generated using the method of claim 1.

31. An influenza virus-like particle (VLP) comprising the non-naturally occurring immunogen generated using the method of claim 1.

32. A immunogenic composition or vaccine comprising the virus-like particle (VLP) of claim 31.

33. A composition comprising the immunogen, vaccine, or VLP of claim 29 and a pharmaceutically acceptable carrier, excipient, or vehicle.

34. (canceled)

35. A method of generating an immune response in a subject, the method comprising administering to the subject an effective amount of an immunogen generated using the method of claim 1.

36. A method of generating an immune response in a subject, the method comprising administering to the subject an effective amount of the immunogen of claim 29.

37. A method of generating an immune response in a subject, the method comprising administering to the subject an effective amount of the immunogenic composition or vaccine of claim 30.

38. A method of generating an immune response in a subject, the method comprising administering to the subject an effective amount of the VLP of claim 31.

39. A method of generating an immune response in a subject, the method comprising administering to the subject an effective amount of the immunogenic composition or vaccine of claim 30.

40. A method of generating an immune response in a subject, the method comprising administering to the subject an effective amount of the composition of claim 33.

41-44. (canceled)


SEQUENCE LISTING (4 sequences)

SEQ ID NO: 1 - protein, 565 aa, Influenza A virus
MKTIIALSYIFCLALGQDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPITCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDVYRDEALNNRFQIKGVELKSGYKDWILWISFAISCFLLCVVLLGFIMWACQRGNIRCNICI

SEQ ID NO: 2 - DNA, 1720 bp, Influenza A virus
attaatcatgaagaccatcattgctttgagctacattttctgtctggctctcggccaagaccttccaggaaatgacaacagcacagcaacgctgtgcctgggacatcatgcggtgccaaacggaacactagtgaaaacaatcacagatgatcagattgaagtgactaatgctactgagctagttcagagctcctcaacggggaaaatatgcaacaatcctcatcgaatccttgatggaatagactgcacactgatagatgctctattgggggaccctcattgtgatgtttttcaaaatgagacatgggaccttttcgttgaacgcagcaaagctttcagcaactgttacccttatgatgtgccagattatgcctcccttaggtcactagttgcctcgtcaggcactctggagtttatcactgagggtttcacttggactggggtcactcagaatgggggaagcaatgcttgcaaaaggggacctggtagcggttttttcagtagactgaactggttgaccaaatcaggaagcacatatccagtgctgaacgtgactatgccaaacaatgacaattttgacaaactatacatttggggggttcaccacccgagcacgaaccaagaacaaaccagcctgtatgttcaagcatcagggagagtcacagtctctaccaggagaagccagcaaactataatcccgaatatcgggtccagaccctgggtaaggggtctgtctagtagaataagcatctattggacaatagttaagccgggagacgtactggtaattaatagtaatgggaacctaatcgctcctcggggttatttcaaaatgcgcactgggaaaagctcaataatgaggtcagatgcacctattgatacctgtatttctgaatgcatcactccaaatggaagcattcccaatgacaagccctttcaaaacgtaaacaagatcacatatggagcatgccccaagtatgttaagcaaaacaccctgaagttggcaacagggatgcggaatgtaccagagaaacaaactagaggcctattcggcgcaatagcaggtttcatagaaaatggttgggagggaatgatagacggttggtacggtttcaggcatcaaaattctgagggcacaggacaagcagcagatcttaaaagcactcaagcagccatcgaccaaatcaatgggaaattgaacagggtaatcgagaagacgaacgagaaattccatcaaatcgaaaaggaattctcagaagtagaagggagaattcaggacctcgagaaatacgttgaagacactaaaatagatctctggtcttacaatgcggagcttcttgtcgctctggagaatcaacatacaattgacctgactgactcggaaatgaacaagctgtttgaaaaaacaaggaggcaactgagggaaaatgctgaagacatgggcaatggttgcttcaaaatataccacaaatgtgacaacgcttgcatagagtcaatcagaaatgggacttatgaccatgatgtatacagagacgaagcattaaacaaccggtttcagatcaaaggtgttgaactgaagtctggatacaaagactggatcctgtggatttcctttgccatatcatgctttttgctttgtgttgttttgctggggttcatcatgtgggcctgccagagaggcaacattaggtgcaacatttgcatttgagtgtattagtaa

SEQ ID NO: 3 - protein, 566 aa, Influenza A virus
MKTIIALSYILCLVFAQKLPGNDNSTATLCLGHHAVPNGTILKTITNDQIEVTNATELVQSSSTGGICDSPHQILDGENCTLIDAWLGDPQCDGFQNKKWDLFVERSKAYSNCYPYDVPDYASLRSLVASSGTLEFNNESFNWTGVTQNGTSSACKRSSNNSFFSRLNWLTHLKFKYPALNVTMPNNEKFDKLYIWGVHHPGTDNDQISLYAQASGRITVSTKRSQQTVIPNIGSRPRARDIPSRISIYWTIVKPGDILAINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAINQINGKLNRLIGKTNEKFHQIEKEFSEVEGRIQDLEKYVEDTKIDLWSYNAELLVALENAHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 4 - protein, 566 aa, Influenza A virus
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASTTTLEFNNESFNWTGVTQNGTSSACKRRRNNSFFSRLNWLTHLKFKYPALNVTMPNNEKFDKLYIWGVHHPGTDNDQIFLYAQASGRITVSTKRSQQTVIPNIGSRPRVRNIPSRISIYWTIAKPGDILLINSTGNLIVPRGYFKIRSGKSSIMRSDAPIGKCNSECITPNGSIPNDKPFQNVNRITYAACPRYVKQNTLKLATGMRNVPEKQTRGIFGAIAGFIENGWEGMVDGWYGFRHQNSEGIGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDIGNGCFKIYHKCDNACIGSIRNGTYDHDVYRDEALNNRFQIKGVELKSGYKDWILWISFAISCFLLCVALLGFIMWACQKGNIRCNICI